SEQUENCE LISTING (2 sequences)

SEQ ID NO: 1 - DNA, 3958 bp, homo sapiens
gtgctgcctcgtctgaggggacaggaggatcaccctcttcgtcgcttcggccagtgtgtcgggctgggccctgacaagccacctgaggagaggctcggagccgggcccggaccccggcgattgccgcccgcttctctctagtctcacgaggggtttcccgcctcgcacccccacctctggacttgcctttccttctcttctccgcgtgtggagggagccagcgcttaggccggagcgagcctgggggccgcccgccgtgaagacatcgcggggaccgattcaccatggagggcgccggcggcgcgaacgacaagaaaaagataagttctgaacgtcgaaaagaaaagtctcgagatgcagccagatctcggcgaagtaaagaatctgaagttttttatgagcttgctcatcagttgccacttccacataatgtgagttcgcatcttgataaggcctctgtgatgaggcttaccatcagctatttgcgtgtgaggaaacttctggatgctggtgatttggatattgaagatgacatgaaagcacagatgaattgcttttatttgaaagccttggatggttttgttatggttctcacagatgatggtgacatgatttacatttctgataatgtgaacaaatacatgggattaactcagtttgaactaactggacacagtgtgtttgattttactcatccatgtgaccatgaggaaatgagagaaatgcttacacacagaaatggccttgtgaaaaagggtaaagaacaaaacacacagcgaagcttttttctcagaatgaagtgtaccctaactagccgaggaagaactatgaacataaagtctgcaacatggaaggtattgcactgcacaggccacattcacgtatatgataccaacagtaaccaacctcagtgtgggtataagaaaccacctatgacctgcttggtgctgatttgtgaacccattcctcacccatcaaatattgaaattcctttagatagcaagactttcctcagtcgacacagcctggatatgaaattttcttattgtgatgaaagaattaccgaattgatgggatatgagccagaagaacttttaggccgctcaatttatgaatattatcatgctttggactctgatcatctgaccaaaactcatcatgatatgtttactaaaggacaagtcaccacaggacagtacaggatgcttgccaaaagaggtggatatgtctgggttgaaactcaagcaactgtcatatataacaccaagaattctcaaccacagtgcattgtatgtgtgaattacgttgtgagtggtattattcagcacgacttgattttctcccttcaacaaacagaatgtgtccttaaaccggttgaatcttcagatatgaaaatgactcagctattcaccaaagttgaatcagaagatacaagtagcctctttgacaaacttaagaaggaacctgatgctttaactttgctggccccagccgctggagacacaatcatatctttagattttggcagcaacgacacagaaactgatgaccagcaacttgaggaagtaccattatataatgatgtaatgctcccctcacccaacgaaaaattacagaatataaatttggcaatgtctccattacccaccgctgaaacgccaaagccacttcgaagtagtgctgaccctgcactcaatcaagaagttgcattaaaattagaaccaaatccagagtcactggaactttcttttaccatgccccagattcaggatcagacacctagtccttccgatggaagcactagacaaagttcacctgagcctaatagtcccagtgaatattgtttttatgtggatagtgatatggtcaatgaattcaagttggaattggtagaaaaactttttgctgaagacacagaagcaaagaacccattttctactcaggacacagatttagacttggagatgttagctccctatatcccaatggatgatgacttccagttacgttccttcgatcagttgtcaccattagaaagcagttccgcaagccctgaaagcgcaagtcctcaaagcacagttacagtattccagcagactcaaatacaagaacctactgctaatgccaccactaccactgccaccactgatgaattaaaaacagtgacaaaagaccgtatggaagacattaaaatattgattgcatctccatctcctacccacatacataaagaaactactagtgccacatcatcaccatatagagatactcaaagtcggacagcctcaccaaacagagcaggaaaaggagtcatagaacagacagaaaaatctcatccaagaagccctaacgtgttatctgtcgctttgagtcaaagaactacagttcctgaggaagaactaaatccaaagatactagctttgcagaatgctcagagaaagcgaaaaatggaacatgatggttcactttttcaagcagtaggaattggaacattattacagcagccagacgatcatgcagctactacatcactttcttggaaacgtgtaaaaggatgcaaatctagtgaacagaatggaatggagcaaaagacaattattttaataccctctgatttagcatgtagactgctggggcaatcaatggatgaaagtggattaccacagctgaccagttatgattgtgaagttaatgctcctatacaaggcagcagaaacctactgcagggtgaagaattactcagagctttggatcaagttaactgagctttttcttaatttcattcctttttttggacactggtggctcactacctaaagcagtctatttatattttctacatctaattttagaagcctggctacaatactgcacaaacttggttagttcaatttttgatcccctttctacttaatttacattaatgctcttttttagtatgttctttaatgctggatcacagacagctcattttctcagttttttggtatttaaaccattgcattgcagtagcatcattttaaaaaatgcacctttttatttatttatttttggctagggagtttatccctttttcgaattatttttaagaagatgccaatataatttttgtaagaaggcagtaacctttcatcatgatcataggcagttgaaaaatttttacaccttttttttcacattttacataaataataatgctttgccagcagtacgtggtagccacaattgcacaatatattttcttaaaaaataccagcagttactcatggaatatattctgcgtttataaaactagtttttaagaagaaattttttttggcctatgaaattgttaaacctggaacatgacattgttaatcatataataatgattcttaaatgctgtatggtttattatttaaatgggtaaagccatttacataatatagaaagatatgcatatatctagaaggtatgtggcatttatttggataaaattctcaattcagagaaatcatctgatgtttctatagtcactttgccagctcaaaagaaaacaataccctatgtagttgtggaagtttatgctaatattgtgtaactgatattaaacctaaatgttctgcctaccctgttggtataaagatattttgagcagactgtaaacaagaaaaaaaaaatcatgcattcttagcaaaattgcctagtatgttaatttgctcaaaatacaatgtttgattttatgcactttgtcgctattaacatcctttttttcatgtagatttcaataattgagtaattttagaagcattattttaggaatatatagttgtcacagtaaatatcttgttttttctatgtacattgtacaaatttttcattccttttgctctttgtggttggatctaacactaactgtattgttttgttacatcaaataaacatcttctgtggaccaggaaaaaaaaaaaaaaaaaaa

SEQ ID NO: 2 - DNA, 16 bp, Artificial Sequence
  Synthetic Oligonucleotide
tggcaagcatcctgta

(19) United States
(12) Patent Application Publication
(10) Pub. No.: US 2012/0122956 A1
(43) Pub. Date: May 17, 2012

(54) METHODS FOR INHIBITING ANGIOGENESIS WITH MULTI-ARM POLYMERIC CONJUGATES OF 7-ETHYL-10-HYDROXYCAMPTOTHECIN

(75) Inventors: Fabio Pastorino, Bogliasco (Genoa) (IT); Mirco Ponzoni, Genoa (IT); Puja Sapra, Edison, NJ (US)

(73) Assignee: ENZON PHARMACEUTICALS, INC., Bridgewater, NJ (US)

(21) Appl. No.: 13/264,567

(22) PCT Filed: Apr. 15, 2010
(86) PCT No.: PCT/US2010/031165

§ 371 Date: Jan. 19, 2012

Related U.S. Application Data
(60) Provisional application No. 61/170,386, filed on Apr. 17, 2009.

(51) Int. Cl. A61K 31/475; A61P 35/00; A61K 31/713

(52) U.S. Cl. 514/44 A; 514/283

(57) ABSTRACT

The present invention relates to methods of inhibiting angiogenesis in mammals. The present invention includes administering polymeric prodrugs of 7-ethyl-10-hydroxycamptothecin to the mammals in need thereof. The present invention also relates to methods of treating a disease associated with angiogenesis in mammals by administering polymeric prodrugs of 7-ethyl-10-hydroxycamptothecin to the mammals in need thereof.

### CROSS-REFERENCE TO RELATED APPLICATION

This application claims the benefit of priority from U.S. Provisional Patent Application Ser. No. 61/170,386, filed Apr. 17, 2009, the contents of which are incorporated herein by reference.

### FIELD OF THE INVENTION

The present invention relates to methods of inhibiting angiogenesis or angiogenic activity by administering polymeric prodrugs of 7-ethyl-10-hydroxycamptothecin. In particular, the invention relates to methods of inhibiting angiogenesis by administering polyethylene glycol conjugates of 7-ethyl-10-hydroxycamptothecin.

### BACKGROUND OF THE INVENTION

Angiogenesis is a natural process in the body involving the formation of new blood vessels. The healthy body controls angiogenesis through maintaining a balance of angiogenesis stimulators and angiogenesis inhibitors.

A variety of diseases and pathological conditions are associated with angiogenesis, either insufficient angiogenesis or excessive angiogenesis. Recently, angiogenesis-based therapeutic approaches have been developed to treat diseases by inhibiting or stimulating angiogenesis. Pro-angiogenic therapies treat diseases such as coronary artery disease, peripheral arterial disease, stroke, wound healing, etc. by using angiogenic growth factors to promote angiogenesis. Anti-angiogenic therapies treat diseases by employing angiogenic inhibitors to block or slow down angiogenesis. For example, various attempts to treat cancer and metastasis use angiogenesis inhibitors, since angiogenesis plays an important role in tumor growth and metastasis, and tumors have more blood vessels relative to normal tissues. A list of known angiogenesis inhibitors includes, for example, angioarrestin, angiostatin (plasminogen fragment), antiangiogenic antithrombin III, cartilage-derived inhibitor (CDI), CD59 complement fragment, endostatin (collagen XVIII fragment), fibronectin fragment, gro-beta, heparinases, heparin hexasaccharide fragment, human chorionic gonadotropin (hCG), interferon alpha/beta/gamma, interferon inducible protein (IP-10), interleukin-12, Kringle 5 (K5; plasminogen fragment), metalloproteinase inhibitors (TIMPs), 2-methoxyestradiol, placental ribonuclease inhibitor, plasminogen activator inhibitor, platelet factor-4 (PF4), prolactin 16 kD fragment, proliferin-related protein (PRP), retinoids, tetrahydrocortisol-S, thrombospondin-1 (TSP-1), transforming growth factor-beta (TGF-b), vasculostatin, vasostatin (calreticulin fragment) and oltipraz [(5-2-pyrazinyl)-4-methyl-1,2-dithiol-3-thione]. The FDA has approved angiogenic inhibitors such as bevacizumab (Avastin®), pegaptanib (Macugen®) for the treatment of certain cancers.

Unfortunately, the known angiogenesis inhibitors prolong survival in patients, but they do not necessarily cure diseases. Thus, patients need to take antiangiogenic agents over a long period, and such long term treatment with angiogenic inhibitors could have adverse effects on the immune system, reproductive system, heart, and so forth.

Thus, there continues to be a need for improved agents and methods for inhibiting angiogenesis. The present invention addresses this need.

### SUMMARY OF THE INVENTION

In one aspect of the present invention, there is provided a method of inhibiting angiogenesis or angiogenic activity in a mammal. The method includes administering an effective amount of a compound of Formula (I):

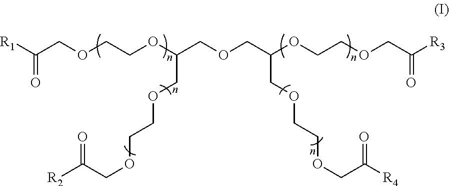

wherein

R₁, R₂, R₃ and R₄ are independently OH or

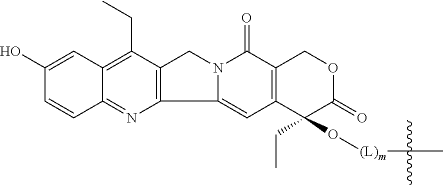

wherein
L is a bifunctional linker, and each L is the same or different when (m) is equal to or greater than 2;
(m) is 0 or a positive integer; and
(n) is a positive integer;
provided that R₁, R₂, R₃ and R₄ are not all OH;
   or a pharmaceutically acceptable salt thereof to the mammal.

In one particular aspect of the invention, the employed polymeric prodrugs of 7-ethyl-10-hydroxycamptothecin include four-arm PEG-7-ethyl-10-hydroxycamptothecin conjugates having the structure of

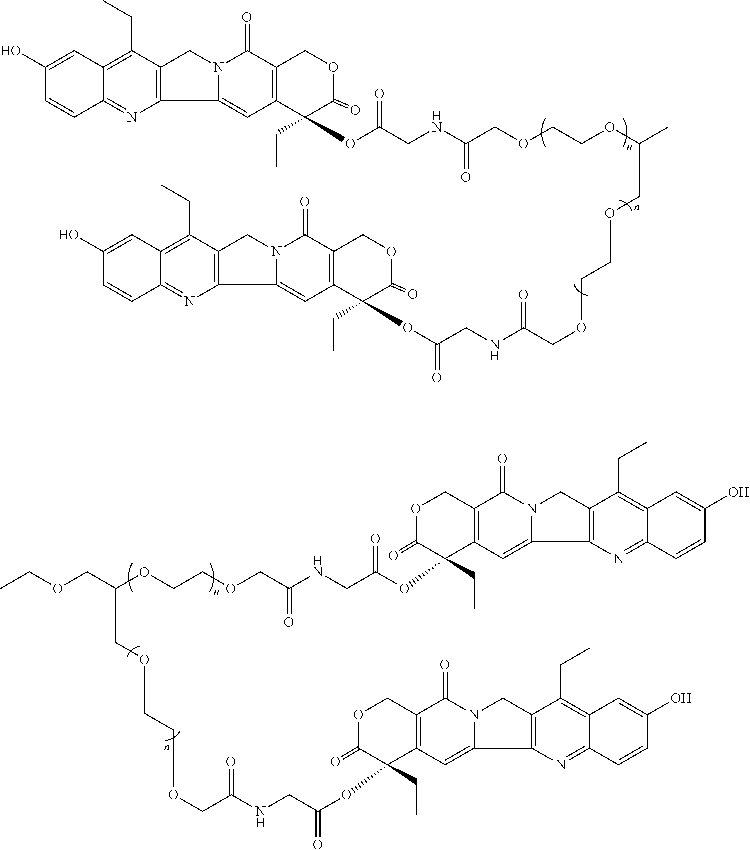

wherein (n) is from about 28 to about 341, preferably from about 114 to about 239, and more preferably about 227.

In another aspect, the present invention provides a method of treating a disease or disorder associated with angiogenesis, as well as a method of inhibiting the growth of an angiogenesis-dependent cell in a mammal.

In yet another aspect, the present invention provides a method of inducing or promoting apoptosis in mammals.

In yet another aspect, the present invention provides a method of delivering 7-ethyl-10-hydroxycomptothecin to a cell in a mammal. The method includes:

(a) forming a polymeric conjugate of 7-ethyl-10-hydroxycomptothecin or a pharmaceutically acceptable salt thereof; and

(b) administering the conjugate or the pharmaceutically acceptable salt thereof to a mammal in need thereof.

In a further aspect, the method of the present invention is conducted wherein the compound of Formula (I), or a pharmaceutically acceptable salt thereof, is administered in combination with an antisense HIF-1α oligonucleotide or a pharmaceutically acceptable salt thereof.

One advantage of the inventive method is that the present invention can be performed in combination with other types of treatments to provide additive effect. For example, the present invention can be conducted in combination with radiotherapy or with administration of one or more additional therapeutic agent(s), concurrently or sequentially.

Another advantage is that the present invention is effective in the control of cancers with poor prognosis (i.e. lymphomas) since the present invention inhibits angiogenesis and also downregulates HIF-1α expression. HIF-1α expression is considered to be correlated with drug resistance and overall poor treatment outcome.

Further advantages will be apparent from the following description and drawings.

For purposes of the present invention, the term “residue” shall be understood to mean that portion of a compound, to which it refers, e.g., 7-ethyl-10-hydroxycamptothecin, amino acid, etc. that remains after it has undergone a substitution reaction with another compound.

For purposes of the present invention, the term “polymeric containing residue” or “PEG residue” shall each be understood to mean that portion of the polymer or PEG which remains after it has undergone a reaction with, e.g., an amino acid, 7-ethyl-10-hydroxycamptothecin-containing compounds.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated number of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkloxy group.

For purposes of the present invention, the term “amino” refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “halogen’ or “halo” refers to fluorine, chlorine, bromine, and iodine.

For purposes of the present invention, the term “heteroatom” refers to nitrogen, oxygen, and sulfur.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

For purposes of the present invention, “positive integer” shall be understood to include an integer equal to or greater than 1 (e.g., 1, 2, 3, 4, 5, 6) and as will be understood by those of ordinary skill to be within the realm of reasonableness by the artisan of ordinary skill.

For purposes of the present invention, use of phrases such as “decreased”, “reduced”, “diminished”, or “lowered” includes at least a 10% change in pharmacological activity with greater percentage changes being preferred for reduction in angiogenesis or levels of angiogenesis-associated gene expression. For instance, the change may also be greater than 25%, 35%, 45%, 55%, 65%, or other increments greater than 10%, or the range may be in a range from 25% through 99%.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art. An effective amount for each mammal or human patient to be treated is readily determined by the artisan in a range that provides a desired clinical response while avoiding undesirable effects that are inconsistent with good practice. Dose ranges are provided hereinbelow.

For purposes of the present invention, the terms “cancer” and “tumor” are used interchangeably, unless otherwise indicated. “Cancer” encompasses malignant and/or metastatic cancer, unless otherwise indicated. Preferably, the term caner includes vascularized solid cancer.

For purposes of the present invention, “regulating angiogenesis’ shall be understood to mean that angiogenesis is effected in a desired way by the treatment described herein. This includes, inhibiting, blocking, reducing, stimulating, inducing, etc., the formation of blood vessels.

For purposes of the present invention, “inhibition of angiogenesis” shall be understood to mean reduction, amelioration or prevention of blood vessel formation or angiogenesis-associated disease realized in patients after completion of the therapy described herein, as compared to mammals (e.g., patients) who have not received the treatment described herein. In one embodiment, successful treatment shall be deemed to occur when at least 10% or preferably 20%, more preferably 30% or higher (i.e., 40%, 50%) decrease in markers contemplated by the artisan in the field is realized when compared to that observed in the absence of the treatment described herein. Useful systems for determining changes in angiogenesis include chicken chorioallantoic membrane (CAM) assay. Other systems include bovine capillary endothelial (BCE) cell assay (e.g., U.S. Pat. No. 6,024,688), HUVEC (human umbilical cord vascular endothelial cell) growth inhibition assay (e.g., U.S. Pat. No. 6,060,449), corneal angiogenesis assay, aortic ring assay and intravital microscopy. In alternatives, successful treatment shall be deemed to occur when at least 10% or preferably 20%, more preferably 30% or higher (i.e., 40%, 50%) decrease in expression of HIF-1α, HIF-2α, VEGF, CD31, MMP-2 or MMP-9, when compared to that observed in the absence of the treatment described herein.

For purposes of the present invention, the terms, “nucleic acid” or “nucleotide” apply to deoxyribonucleic acid (“DNA”), ribonucleic acid, (“RNA”) whether single-stranded or double-stranded, unless otherwise specified, and any chemical modifications thereof.

### BRIEF DESCRIPTION OF THE DRAWINGS

FIG. 1 schematically illustrates a reaction scheme for preparing four-arm polyethylene glycol acids described in Examples 1-2.

FIG. 2 schematically illustrates a reaction scheme for preparing 4arm-PEG-Gly-(7-ethyl-10-hydroxycamptothecin) described in Examples 3-7.

FIG. 3 schematically illustrates a reaction scheme for preparing 4arm-PEG-Ala-(7-ethyl-10-hydroxycamptothecin) described in Examples 8-12.

FIG. 4 schematically illustrates a reaction scheme for preparing 4arm-PEG-Met-(7-ethyl-10-hydroxycamptothecin) described in Examples 13-16.

FIG. 5 schematically illustrates a reaction scheme for preparing 4arm-PEG-Sar-(7-ethyl-10-hydroxycamptothecin) described in Examples 17-21.

FIG. 6 shows the stability of 4arm-PEG-Gly-(7-ethyl-10-hydroxycamptothecin) as described in Example 24.

FIG. 7 shows the effect of pH on stability of 4arm-PEG-Gly-(7-ethyl-10-hydroxycamptothecin) as described in Example 24.

FIGS. 8A and 8B show pharmacokinetic profiles of 4arm-PEG-Gly-(7-ethyl-10-hydroxy-camptothecin) as described in Example 25.

FIG. 9A provides photomicrographs that illustrate the results of chorioallantoic membrane (“CAM”) assays for blood vessel growth conducted using biopsy samples according to Example 26.

FIG. 9B illustrates a comparison of CD31-positive microvessels in treated and control samples.

FIG. 10A provides images that illustrate relative expression of VEGF and CD31 in biopsy samples prepared according to Example 27.

FIG. 10B illustrates the relative percentage expression of VEGF and CD31 in biopsy samples prepared according to Example 27.

FIG. 10C provides images illustrate relative expression of MMP-2 and MMP-9 in biopsy samples prepared according to Example 27.

FIG. 10D illustrates the relative percentage expression of MMP-2 and MMP-9 in biopsy samples prepared according to Example 27.

FIG. 11A provides photomicrographs that illustrate enhanced TUNEL and histone H2ax immunotstaining on biopsy samples prepared according to Examples 27-28. In FIG. 11A, light areas indicate areas with more apoptotic cells.

FIGS. 11B and 11C illustrate the relative percentage of TUNEL (FIG. 11B) and H2ax immunostaining (FIG. 11C) on biopsy samples prepared according to Examples 27-28.

FIG. 12A illustrates the percentage change from baseline of HIF-1α expression in a human glioma xenograft model with a single dose of compound 9, according to Example 29. The open bars (rectangles) indicate zero hours; the gray bars indicate 48 hours; and the black bars indicate 120 hours.

FIG. 12B provides photographs that illustrates relative HIF-1-dependent luciferase expression at baseline and at 120 hours, with a single dose (qdx1) of compound 9, in the U251-HRE xenografts according to Example 29. FIG. 12C illustrates the percentage change from baseline of HIF-1α expression in a human glioma xenograft model with multiple doses (q2d×3) of compound 9, according to Example 29. The open bars (rectangles) indicate zero hours; the gray bars indicate 48 hours; and the black bars indicate 120 hours.

FIG. 12D provides photographs that illustrate relative HIF-1-dependent luciferase expression at baseline and at 120 hours, with multiple doses (q2d×3) of compound 9, in the U251-HRE xenografts according to Example 29.

FIG. 13 illustrates the reduction in tumor mass in the xenografted mice recorded in the tests according to Example 29, from zero to 125 hours of treatment.

FIG. 14A provides western blot images that illustrate relative HIF-2α expression in the samples prepared according to Example 30.

FIG. 14B provides western blot images that illustrate relative HIF-1α expression in the samples prepared according to Example 30.

FIG. 14C provides western blot images that illustrate relative HIF-1α expression in the samples prepared according to Example 30.

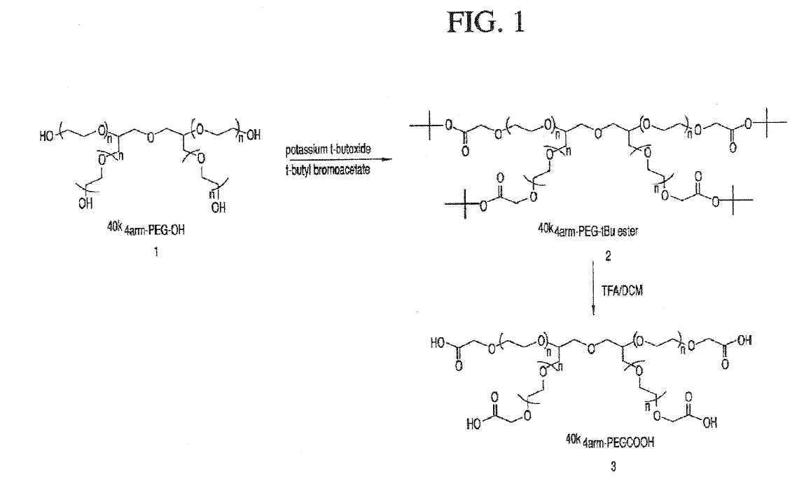

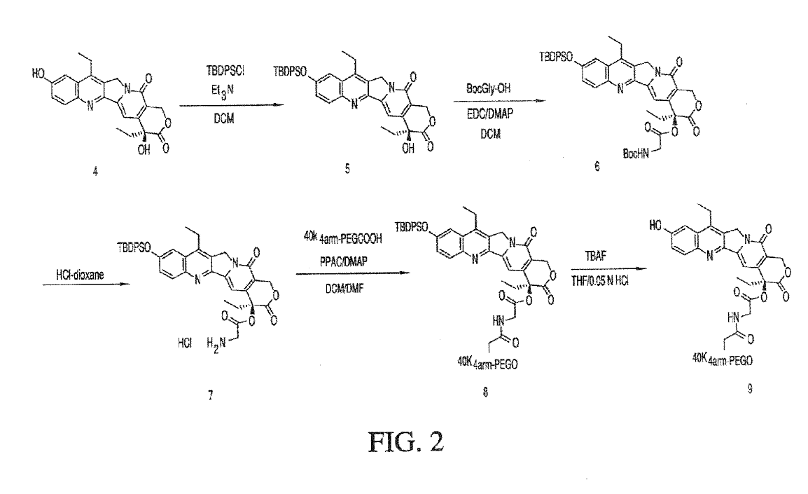

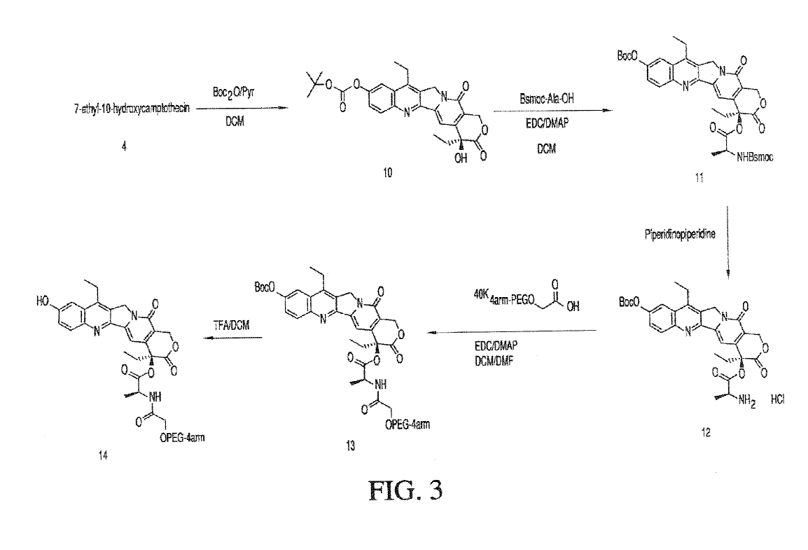

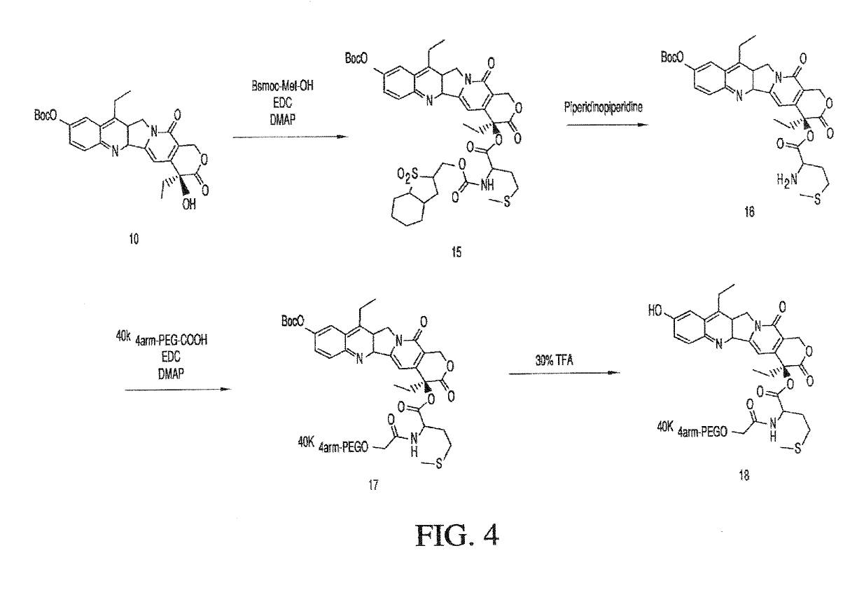

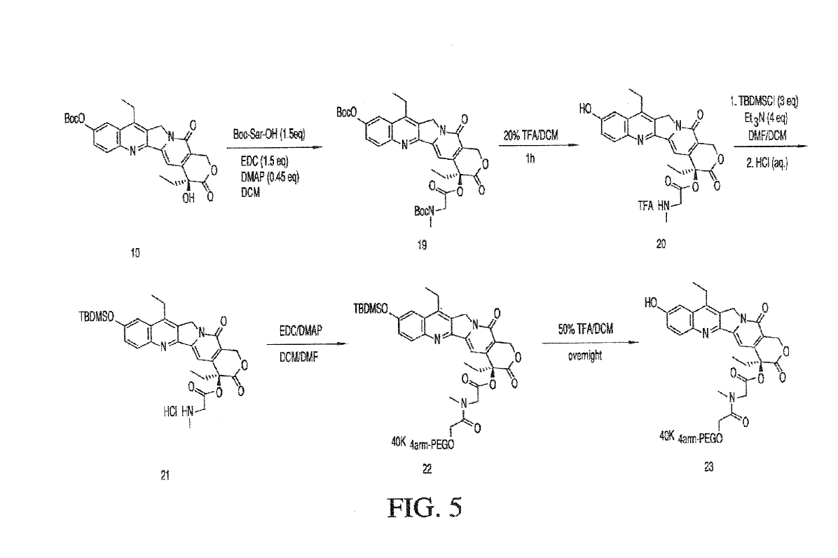

### DETAILED DESCRIPTION OF THE INVENTION

### A. Overview

In one aspect of the invention, there are provided methods of inhibiting angiogenesis or angiogenic activity in a mammal. The method includes:

administering an effective amount of a compound of Formula (I):

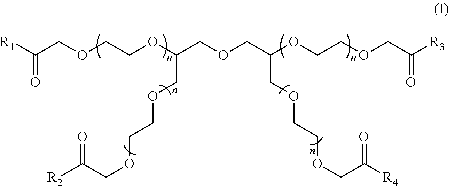

wherein

R₁, R₂, R₃ and R₄ are independently OH or

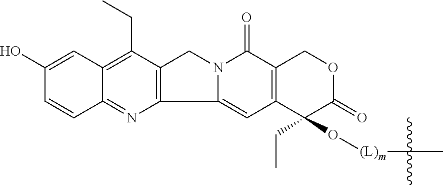

wherein
L is a bifunctional linker;
(m) is 0 or a positive integer, wherein each L is the same or different when (m) is equal to or greater than 2; and
(n) is a positive integer;
provided that R₁, R₂, R₃ and R₄ are not all OH;
   or a pharmaceutically acceptable salt thereof to said mammal.

In one preferred embodiment, the method includes a compound of Formula (I) as part of a pharmaceutical composition, and R₁, R₂, R₃ and R₄ are all:

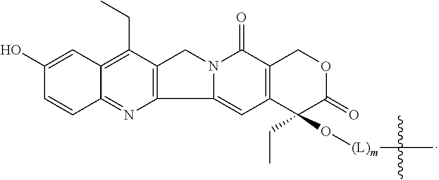

In more preferred aspect, the method includes administering a compound of Formula (Ia):

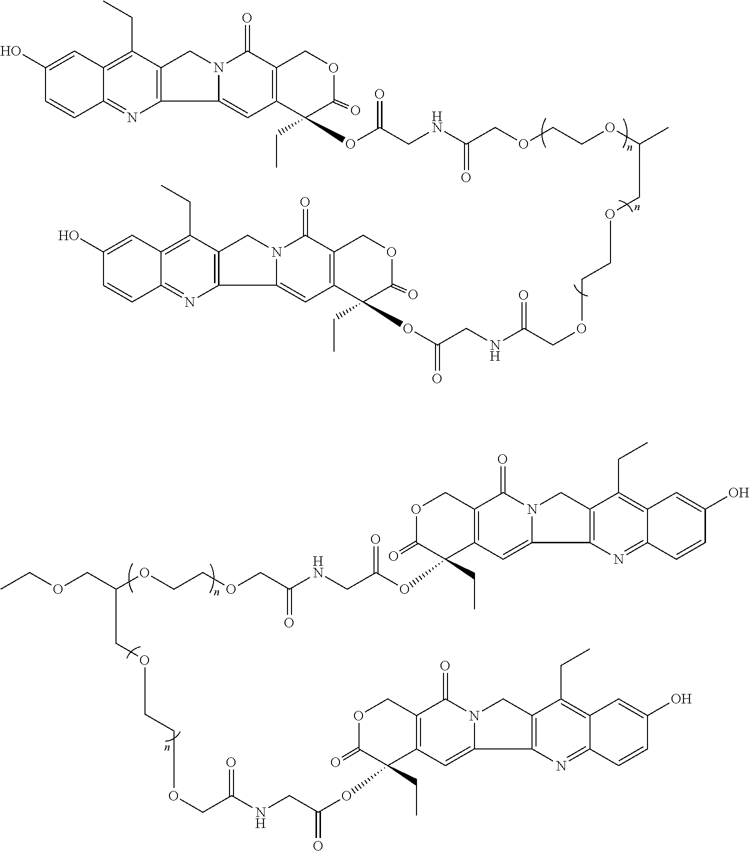

wherein (n) is about 227 so that the polymeric portion of the compound has the total number average molecular weight of about 40,000 daltons.

The compound of Formula (I) employed in the present invention has the angiogenic activity in cells and/or tissues. In certain embodiments, the present invention is conducted wherein the compound described herein inhibits a tumoral angiogenesis or tumor-dependent angiogenesis.

In another aspect of the invention, the present invention provides methods of treating a disease or disorder associated with angiogenesis in a mammal. The method includes administering an effective amount of a compound of Formula (I):

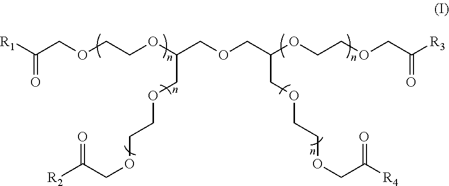

wherein

R₁, R₂, R₃ and R₄ are independently OH or

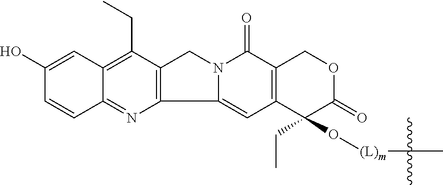

wherein
L is a bifunctional linker;
(m) is 0 or a positive integer, wherein each L is the same or different when (m) is equal to or greater than 2; and
(n) is a positive integer;
provided that R₁, R₂, R₃ and R₄ are not all OH;
   or a pharmaceutically acceptable salt thereof to said mammal.

In one embodiment, the methods of the present invention described herein are conducted wherein the diseases or disorders associated with angiogenesis include neoplastic diseases, atherosclerosis, restenosis, rheumatoid arthritis, Crohn's disease, diabetic retinopathy, psoriasis, endometriosis, macular degeneration, neovascular glaucoma, and adiposity. Pathological conditions which involve excessive angiogenesis benefit from inhibition of angiogenesis. These methods preferably include the step of identifying a patient having such a disease or disorder.

In another embodiment, the present invention provides a method of treating the growth or metastasis of an angiogenesis-dependent cancer in a mammal by administering the compound of Formula (I) described herein or a pharmaceutically acceptable salt thereof to a mammal. For example, the angiogenesis-dependent cancer includes solid tumors, colorectal cancer, pancreatic cancer, lung cancer, small cell lung cancer, non-small cell lung cancer (NSCLC), stomach cancer, gastrointestinal stromal tumor (GIST), esophageal cancer, prostate cancer, kidney (renal) cancer, liver cancer, lymphomas, leukemia, acute lymphocytic leukemia (ALL), melanoma, multiple myeloma, acute myeloid leukimia (AML), breast cancer, bladder cancer, glioblastoma, ovarian cancer, non-Hodgkin's lymphoma, anal cancer, neuroblastoma, head and neck cancer. The angiogenesis-dependent cancer includes metastatic cancer (e.g., metastatic colorectal cancer, metastatic breast cancer). In certain embodiments, the therapy with the compound of Formula (I) can be administered with radiation therapy concurrently or sequentially.

In yet another aspect, the present invention provides a method of inhibiting the growth of an angiogenesis-dependent cell in a mammal. The method includes administering an effective amount of the compound of Formula (I) or a pharmaceutically acceptable salt thereof to the mammal. Alternatively, the method is conducted by delivering the compound of Formula (I) or a pharmaceutically acceptable salt thereof to cells and tissues in the mammal in need thereof. In certain aspects, the cells are cancerous cells.

In a still further aspect, the present invention provides a method of treating a disease or disorder associated with higher levels of HIF-1α gene (e.g., gene expression) or protein, compared to that observed in a mammal without the disease. The method includes administering the compound of Formula (I) or a pharmaceutically acceptable salt thereof to the mammal. The method can be conducted wherein the compound of Formula (I) or a pharmaceutically acceptable salt thereof is administered in combination with an antisense HIF-1α olignucleotide.

In a still further embodiment of the invention, the present invention provides a method of treating a disease or a disorder associated with higher levels of gene or protein expression associated with angiogenesis (e.g., HIF-1 alpha, HIF-2 beta, VEGF), compared to that observed in a mammal with normal expression of such gene or protein (or without excessive expression of such gene or protein). The methods are useful in the treatment of patients with abnormal expression of gene or protein associated with angiogenesis. The methods include:
(a) determining levels of gene or protein expression associated with angiogenesis in a patient having a disease or a disorder associated with higher levels of such gene or protein;
(b) administering a compound of Formula (I) to a patient in need thereof.

In a still further embodiment of the invention, the present invention provides a method of adjusting/optimizing dosing for treating a disease or a disorder associated with higher levels of gene or protein expression associated with angiogenesis (e.g., HIF-1 alpha, HIF-2 beta, VEGF), compared to that observed in a mammal with normal expression of such gene or protein (or without excessive expression of such gene or protein). The methods include:
(a) administering a compound of Formula (I) to a patient in need thereof;
(b) determining levels of gene or protein expression associated with angiogenesis; and
(c) adjusting dosing of the compound of Formula (I).

In a still further aspect of the invention, the present invention provides a method of inhibiting HIF-1α induced blood vessel formation or invasion in a mammal. The method includes administering the compound of Formula (I) or pharmaceutically acceptable salt thereof to the mammal. In a still further aspect, the method can be conducted in combination with an antisense HIF-1α olignucleotide.

In an alternative aspect, the present invention provides a method of reducing a vascular network in a mammal having a cancer. The method includes administering the compound of Formula (I) or pharmaceutically acceptable salt thereof to the mammal having a cancer. The method described herein reduces the development of a vascularized solid tumor or metastasis from a primary tumor. In a still further aspect, the method can be conducted in combination with an antisense HIF-1α olignucleotide.

In yet another aspect, the present invention provides a method of inducing or promoting apoptosis in a mammal. The method includes administering an effective amount of a compound of Formula (I) or a pharmaceutically acceptable salt thereof to the mammal. The method induces or increases apoptosis of tumor cells.

In yet another aspect, the present invention provides a method of delivering 7-ethyl-10-hydroxycomptothecin to a cell in a mammal. The method includes:

(a) forming a polymeric conjugate of 7-ethyl-10-hydroxycomptothecin or a pharmaceutically acceptable salt thereof; and

(b) administering the conjugate or the pharmaceutically acceptable salt thereof to a mammal in need thereof.

In one embodiment, the method is conducted wherein the polymeric conjugate includes a polyalkylene oxide. Preferably, the method employs the compound of Formula (I).

In a further aspect, the present invention is conducted wherein the compound of Formula (I) or an pharmaceutically acceptable salt thereof is administered in combination with an antisense HIF-1α oligonucleotide or an pharmaceutically acceptable salt thereof concurrently or sequentially.

In a still further aspect, the present invention provides a method of treating a cancer in a mammal. The method is conducted by administering to said mammal:

(i) an effective amount of an antisense HIF-1α oligonucleotide of about 8 to 50 nucleotides in length that is complementary to at least 8 consecutive nucleotides set forth in SEQ ID NO: 1 or a pharmaceutically acceptable thereof, wherein the antisense HIF-1α oligonucleotide comprises one or more phosphorothioate internucleotide linkages, and one or more locked nucleic acids; and

(ii) an effective amount of a compound of Formula (Ia)

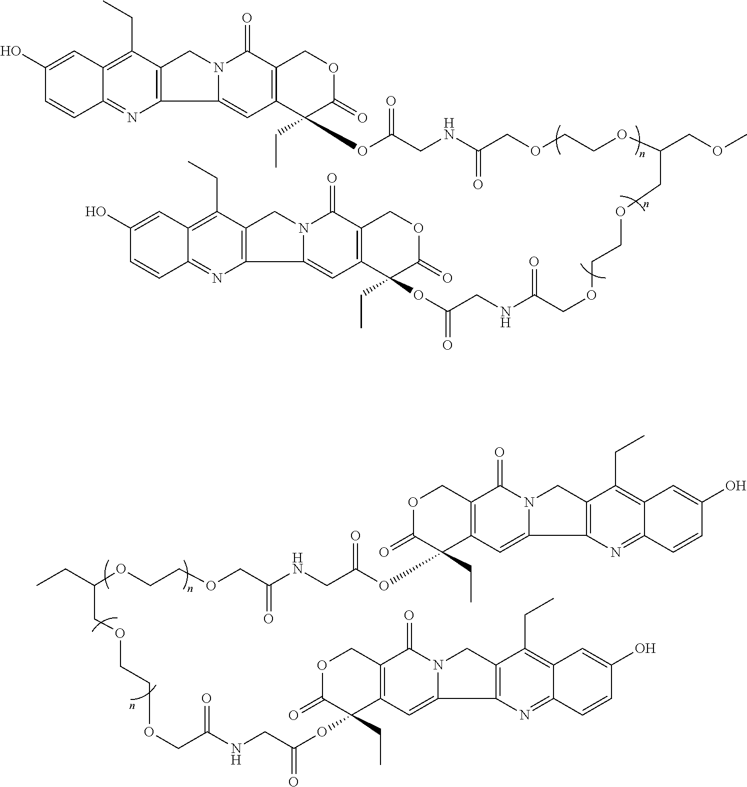

or a pharmaceutically acceptable salt thereof, wherein (n) is about 227 so that the total molecular weight of the polymeric portion of the compound of Formula (Ia) is about 40,000 daltons.

In one preferred embodiment, the antisense HIF-1α oligonucleotide is administered in an amount of from about 4 to about 25 mg/kg/dose, and the compound of Formula (Ia) is administered in an amount of from about 1 mg/m² body surface/dose to about 18 mg/m² body surface/dose, wherein the amount of the compound of Formula (Ia) is the weight of 7-ethyl-10-hydroxycamptothecin included in the compound of Formula (Ia).

In another preferred aspect, the method described herein provides a method of treating an angiogenesis-dependent cancer.

For purposes of the present invention, “inhibition of angiogenesis” shall be understood to mean reduction, amelioration and prevention of the occurrence of angiogenesis (new blood vessel formation) realized in patients as compared to patients which have not received the compound of Formula (I) described herein. In certain aspects, “inhibition of angiogenesis” can be determined by changes in tumor growth, tumor burden and/or metastasis, remission of tumor, or prevention of recurrences of tumor and/or neoplastic growths in patients after completion of treatment with the compounds of Formula (I).

For purposes of the present invention, diseases or disorders associated with angiogenesis contemplated according to the present invention includes conditions in which angiogenesis plays a role in the pathology or progression of the condition, such that inhibition of angiogenesis in a patient having such a condition may delay or prevent the further progression of the condition, or lead to remission or regression of the disease state. In certain aspects, such conditions are associated with abnormal cellular proliferation and growth as in cancer.

For purposes of the present invention, “treatment of tumor/cancer” shall be understood to mean inhibition, reduction, amelioration and prevention of tumor growth, tumor burden and metastasis, remission of tumor, or prevention of recurrences of tumor and/or neoplastic growths realized in patients after completion of anticancer therapy, as compared to patients who have not received anticancer therapy.

Treatment is deemed to occur when a patient achieves positive clinical results. For example, successful treatment of a tumor shall be deemed to occur when at least 10% or preferably 20%, more preferably 30% or higher (i.e., 40%, 50%) decrease in tumor growth including other clinical markers contemplated by the artisan in the field is realized when compared to that observed in the absence of the treatment described herein. Other methods for determining changes in a tumor clinical status resulting from the treatment described herein include: biopsies such as tumor biopsy; immunohistochemistry study using antibody, radioisotope, dye; and complete blood count (CBC).

### B. Compound of Formula (I):

1. Multi-Arm Polymers

The polymeric portion of the compounds described herein includes multi-arm PEG's attached to 20-OH group of 7-ethyl-10-hydroxycamptothecin. In one aspect of the present invention, the polymeric prodrugs of 7-ethyl-10-hydroxy-camptothecin include four-arm PEG, prior to conjugation, having the following structure of

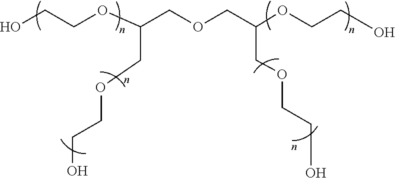

wherein (n) is a positive integer.

The multi-arm PEG's are those described in NOF Corp. Drug Delivery System catalog, Ver. 8, April 2006, the disclosure of which is incorporated herein by reference.

In one preferred embodiment of the invention, the degree of polymerization for the polymer (n) is from about 28 to about 341 to provide polymers having the total number average molecular weight of from about 5,000 Da to about 60,000 Da, and preferably from about 114 to about 239 to provide polymers having the total number average molecular weight of from about 20,000 Da to about 42,000 Da. (n) represents the number of repeating units in the polymer chain and is dependent on the molecular weight of the polymer. In one particularly preferred embodiment of the invention, (n) is about 227 to provide the polymeric portion having the total number average molecular weight of about 40,000 Da.

2. Bifunctional Linkers

In certain preferred aspects of the present invention, bifunctional linkers include an amino acid. The amino acid which can be selected from any of the known naturally-occurring L-amino acids is, e.g., alanine, valine, leucine, isoleucine, glycine, serine, threonine, methionine, cysteine, phenylalanine, tyrosine, tryptophan, aspartic acid, glutamic acid, lysine, arginine, histidine, proline, and/or a combination thereof, to name but a few. In alternative aspects, L can be a peptide residue. The peptide can range in size, for instance, from about 2 to about 10 amino acid residues (e.g., 2, 3, 4, 5, or 6).

Derivatives and analogs of the naturally occurring amino acids, as well as various art-known non-naturally occurring amino acids (D or L), hydrophobic or non-hydrophobic, are also contemplated to be within the scope of the invention. Simply by way of example, amino acid analogs and derivates include: 2-aminoadipic acid, 3-aminoadipic acid, beta-alanine, beta-aminopropionic acid, 2-aminobutyric acid, 4-aminobutyric acid, piperidinic acid, 6-aminocaproic acid, 2-aminoheptanoic acid, 2-aminoisobutyric acid, 3-aminoisobutyric acid, 2-aminopimelic acid, 2,4-aminobutyric acid, desmosine, 2,2-diaminopimelic acid, 2,3-diaminopropionic acid, N-ethylglycine, N-ethylasparagine, 3-hydroxyproline, 4-hydroxyproline, isodesmosine, allo-isoleucine, N-methylglycine or sarcosine, N-methylisoleucine, 6-N-methyllysine, N-methylvaline, norvaline, norleucine, ornithine, and others too numerous to mention, that are listed in 63 Fed. Reg., 29620, 29622, incorporated by reference herein. Some preferred L groups include glycine, alanine, methionine or sarcosine. For example, the compounds can be among:

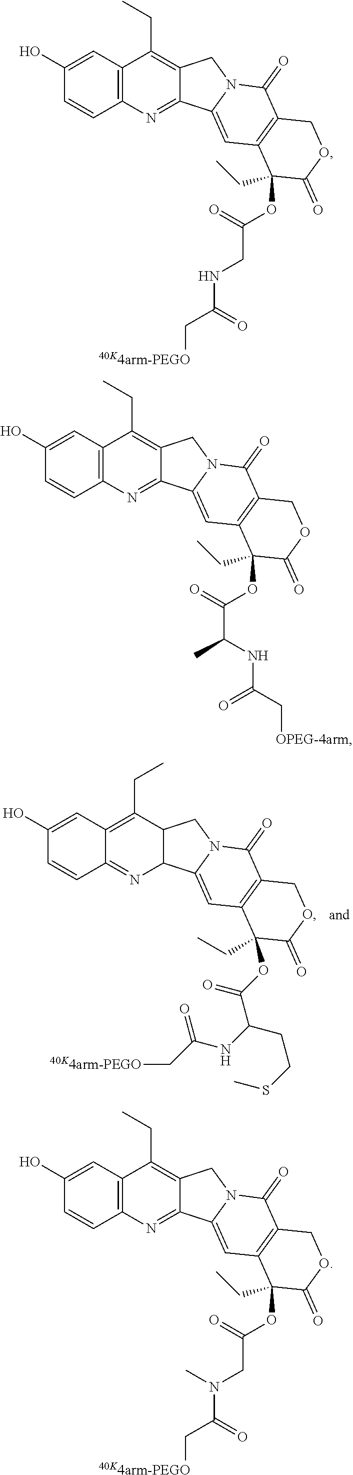

For ease of the description and not limitation, only one arm of the four-arm PEG is shown. One arm, up to four arms of the four-arm PEG can be conjugated with 7-ethyl-10-hydroxy-camptothecin.

More preferably, the treatment described herein employs compounds including a glycine as the linker group (L).

In an alternative aspect of the present invention, L after attachment between the polymer and 7-ethyl-10-hydroxycamptothecin can be selected among:

—[C(═O)]ᵥ(CR₂₂R₂₃)ₜ—,

—[C(═O)]ᵥ(CR₂₂R₂₃)ₜ—O—,

—[C(═O)]ᵥ(CR₂₂R₂₃)ₜNR₂₆—,

—[C(═O)]ᵥO(CR₂₂R₂₃)ₜ—,

—[C(═O)]ᵥO(CR₂₂R₂₃)ₜO—,

—[C(═O)]ᵥO(CR₂₂R₂₃)ₜNR₂₆—,

—[C(═O)]ᵥNR₂₁(CR₂₂R₂₃)ₜ—,

—[C(═O)]ᵥNR₂₁(CR₂₂R₂₃)ₜO—,

—[C(═O)]ᵥNR₂₁(CR₂₂R₂₃)ₜNR₂₆—,

—[C(═O)]ᵥ(CR₂₂R₂₃O)ₜ—,

—[C(═O)]ᵥO(CR₂₂R₂₃O)ₜ—,

—[C(═O)]ᵥNR₂₁(CR₂₂R₂₃O)ₜ—,

—[C(═O)]ᵥ(CR₂₂R₂₃O)ₜ(CR₂₄R₂₅)_{y}—,

—[C(═O)]ᵥO(CR₂₂R₂₃O)ₜ(CR₂₄R₂₅)_{y}—,

—[C(═O)]ᵥNR₂₁(CR₂₂R₂₃O)ₜ(CR₂₄R₂₅)_{y}—,

—[C(═O)]ᵥ(CR₂₂R₂₃O)ₜ(CR₂₄R₂₅)_{y}O—,

—[C(═O)]ᵥ(CR₂₂R₂₃)ₜ(CR₂₄R₂₅O)_{y}—,

—[C(═O)]ᵥO(CR₂₂R₂₃O)ₜ(CR₂₄R₂₅)_{y}O—,

—[C(═O)]ᵥO(CR₂₂R₂₃)ₜ(CR₂₄R₂₅O)_{y}—,

—[C(═O)]ᵥNR₂₁(CR₂₂R₂₃O)ₜ(CR₂₄R₂₅)_{y}O—,

—[C(↑O)]ᵥNR₂₁(CR₂₂R₂₃)ₜ(CR₂₄R₂₅O)_{y}—,

—[C(═O)]ᵥ(CR₂₂R₂₃)ₜO—(CR₂₈R₂₉)_{t′}—,

—[C(═O)]ᵥ(CR₂₂R₂₃)ₜNR₂₆—(CR₂₈R₂₉)_{t′}—,

—[C(═O)]ᵥO(CR₂₂R₂₃)ₜS—(CR₂₈R₂₉)_{t′}—,

—[C(═O)]ᵥO(CR₂₂R₂₃)ₜO—(CR₂₈R₂₉)_{t′}—,

—[C(═O)]ᵥO(CR₂₂R₂₃)ₜNR₂₆—(CR₂₈R₂₉)_{t′}—,

—[C(═O)]ᵥO(CR₂₂R₂₃)ₜS—(CR₂₈R₂₉)_{t′}—,

—[C(═O)]ᵥNR₂₁(CR₂₂R₂₃)ₜO—(CR₂₈R₂₉)_{t′}—,

—[C(═O)]ᵥNR₂₁(CR₂₂R₂₃)ₜNR₂₆—(CR₂₈R₂₉)_{t′}—,

—[C(═O)]ᵥNR₂₁(CR₂₂R₂₃)ₜS—(CR₂₈R₂₉)_{t′}—,

—[C(═O)]ᵥ(CR₂₂R₂₃CR₂₈R₂₉O)ₜNR₂₆—,

—[C(═O)]ᵥ(CR₂₂R₂₃CR₂₈R₂₉O)ₜ—,

—[C(═O)]ᵥO(CR₂₂R₂₃CR₂₈R₂₉O)ₜNR₂₆—,

—[C(═O)]ᵥO(CR₂₂R₂₃CR₂₈R₂₉O)ₜ—,

—[C(═O)]ᵥNR₂₁(CR₂₂R₂₃CR₂₈R₂₉O)ₜNR₂₆—,

—[C(═O)]ᵥNR₂₁(CR₂₂R₂₃CR₂₈R₂₉O)ₜ—,

—[C(═O)]ᵥ(CR₂₂R₂₃CR₂₈R₂₉O)ₜ(CR₂₄R₂₅)_{y}—,

—[C(═O)]ᵥO(CR₂₂R₂₃CR₂₈R₂₉O)ₜ(CR₂₄R₂₅)_{y}—,

—[C(═O)]ᵥNR₂₁(CR₂₂R₂₃CR₂₈R₂₉O)ₜ(CR₂₄R₂₅)_{y}—,

—[C(═O)]ᵥ(CR₂₂R₂₃CR₂₈R₂₉O)ₜ(CR₂₄R₂₅)_{y}O—,

—[C(═O)]ᵥ(CR₂₂R₂₃)ₜ(CR₂₄R₂₅CR₂₈R₂₉O)_{y}—,

—[C(═O)]ᵥ(CR₂₂R₂₃)ₜ(CR₂₄R₂₅CR₂₈R₂₉O)_{y}NR₂₆—,

—[C(═O)]ᵥO(CR₂₂R₂₃CR₂₈R₂₉O)ₜ(CR₂₄R₂₅)_{y}O—,

—[C(═O)]ᵥO(CR₂₂R₂₃)ₜ(CR₂₄R₂₅CR₂₈R₂₉O)_{y}—,

—[C(═O)]ᵥO(CR₂₂R₂₃)ₜ(CR₂₄CR₂₅CR₂₈R₂₉O)_{y}NR₂₆—,

—[C(═O)]ᵥNR₂₁(CR₂₂R₂₃CR₂₈R₂₉O)ₜ(CR₂₄R₂₅)_{y}O—,

—[C(═O)]ᵥNR₂₁(CR₂₂R₂₃)ₜ(CR₂₄R₂₅CR₂₈R₂₉O)_{y}—,

—[C(═O)]ᵥNR₂₁(CR₂₂R₂₃)ₜ(CR₂₄R₂₅CR₂₈R₂₉O)_{y}NR₂₆—,

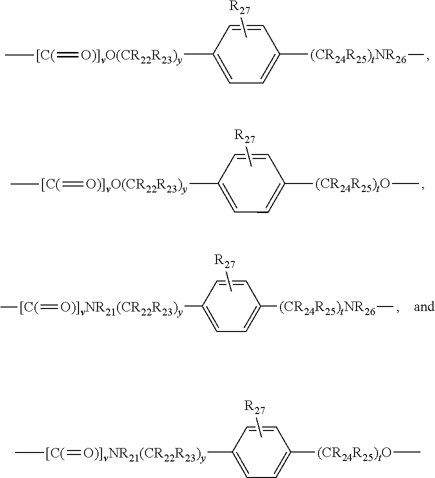

wherein:

R₂₁-R₂₉ are independently selected among hydrogen, amino, substituted amino, azido, carboxy, cyano, halo, hydroxyl, nitro, silyl ether, sulfonyl, mercapto, C₁₋₆ alkylmercapto, arylmercapto, substituted arylmercapto, substituted C₁₋₆ alkylthio, C₁₋₆ alkyls, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy, substituted and arylcarbonyloxy;

(t), (t′) and (y) are independently chosen from zero or a positive integer, preferably from about 1 to about 10 such as 1, 2, 3, 4, 5 and 6; and

(v) is 0 or 1.

In some preferred embodiments, L can include:

—[C(═O)]ᵥ(CH₂)ₜ—,

—[C(═O)]ᵥ(CH₂)ₜ—O—,

—[C(═O)]ᵥ(CH₂)ₜ—NR₂₆—,

—[C(═O)]ᵥO(CH₂)ₜ—,

—[C(═O)]ᵥO(CH₂)ₜO—,

—[C(═O)]ᵥO(CH₂)ₜNH—,

—[C(═O)]ᵥNH(CH₂)ₜ—,

—[C(═O)]ᵥNH(CH₂)ₜO—,

—[C(═O)]ᵥNH(CH₂)ₜNH—,

—[C(═O)]ᵥ(CH₂O)ₜ—,

—[C(═O)]ᵥO(CH₂O)ₜ—,

—[C(═O)]ᵥNH(CH₂O)ₜ—,

—[C(═O)]ᵥ(CH₂O)ₜ(CH₂)_{y}—,

—[C(═O)]ᵥO(CH₂O)ₜH₂)_{y}—,

—[C(═O)]ᵥNH(CH₂O)ₜ(CH₂₅)_{y}—,

—[C(═O)]ᵥ(CH₂O)ₜ(CH₂)_{y}O—,

—[C(═O)]ᵥ(CH₂)ₜ(CH₂O)_{y}—,

—[C(═O)]ᵥO(CH₂O)ₜ(CH₂)_{y}O—,

—[C(═O)]ᵥO(CH₂)ₜ(CH₂O)_{y}—,

—[C(═O)]ᵥNH(CH₂O)ₜ(CH₂)_{y}O—,

—[C(═O)]ᵥNH(CR₂₂R₂₃)ₜ(CH₂O)_{y}—,

—[C(═O)]ᵥ(CH₂)ₜO—(CH₂)_{t′}—,

—[C(═O)]ᵥ(CH₂)ₜNH—(CH₂)_{t′}—,

—[C(═O)]ᵥ(CH₂)ₜS—(CH₂)_{t′}—,

—[C(═O)]ᵥO(CH₂)ₜO—(CH₂)_{t′}—,

—[C(═O)]ᵥO(CH₂)ₜNH—(CH₂)_{t′}—,

—[C(═O)]ᵥO(CH₂)ₜS—(CH₂)_{t′}—,

—[C(═O)]ᵥNH(CR₂₂R₂₃)ₜO—(CH₂)_{t′}—,

—[C(═O)]ᵥNH(CH₂)ₜNH—(CH₂)_{t′}—,

—[C(═O)]ᵥNH(CH₂)ₜS—(CH₂)ₜ—,

—[C(═O)]ᵥ(CH₂CH₂O)ₜNR₂₆—,

—[C(═O)]ᵥ(CH₂CH₂O)ₜ—,

—[C(═O)]ᵥO(CH₂CH₂O)ₜNH—,

—[C(═O)]ᵥO(CH₂CH₂O)ₜ—,

—[C(═O)]ᵥNH(CH₂CH₂O)ₜNH—,

—[C(═O)]ᵥNH(CH₂CH₂O)ₜ—,

—[C(═O)]ᵥ(CH₂CH₂O)ₜ(CH₂)_{y}—,

—[C(═O)]ᵥO(CH₂CH₂O)ₜ(CH₂)_{y}—,

—[C(═O)]ᵥNH(CH₂CH₂O)ₜ(CH₂)_{y}—,

—[C(═O)]ᵥ(CH₂CH₂O)ₜ(CH₂)_{y}O—,

—[C(═O)]ᵥ(CH₂)ₜ(CH₂CH₂O)_{y}—,

—[C(═O)]ᵥ(CH₂)ₜ(CH₂CH₂O)_{y}NH—,

—[C(═O)]ᵥO(CH₂CH₂O)ₜ(CH₂)_{y}O—,

—[C(═O)]ᵥO(CH₂)ₜ(CH₂CH₂O)_{y}—,

—[C(═O)]ᵥO(CH₂)ₜ(CH₂CH₂O)_{y}NH—,

—[C(═O)]ᵥNH(CH₂CH₂O)ₜ(CH₂)_{y}O—,

—[C(═O)]ᵥNH(CH₂)ₜ(CH₂CH₂O)_{y}—,

—[C(═O)]ᵥNH(CH₂)ₜ(CH₂CH₂O)_{y}NH—,

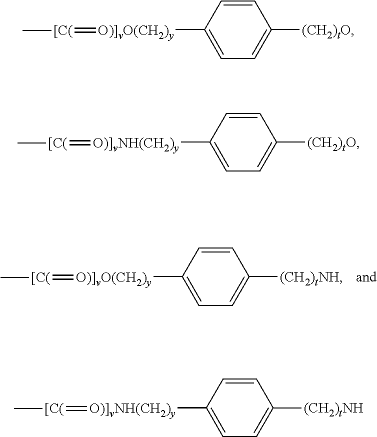

wherein (t), (t′) and (y) are independently chosen from zero or a positive integer, preferably from about 1 to about 10 (e.g., 1, 2, 3, 4, 5, and 6); and

(v) is 0 or 1.

In some aspects of the present invention, the compounds of Formula (I) include from 1 to about 10 units (e.g., 1, 2, 3, 4, 5, or 6) of the bifunctional linker. In some preferred aspects of the present invention, the compounds include one unit of the bifunctional linker and thus (m) is 1.

Additional linkers are found in Table 1 of Greenwald et al. (Bioorganic & Medicinal Chemistry, 1998, 6:551-562), the contents of which are incorporated by reference herein.

3. Synthesis of Prodrugs

Generally, the polymeric prodrugs employed in treatment are prepared by reacting one or more equivalents of an activated multi-arm polymer with, for example, one or more equivalents per active site of amino acid-(20)-7-ethyl-10-hydroxycamptothecin under conditions which are sufficient to effectively cause the amino group to undergo a reaction with the carboxylic acid of the polymer and form a linkage. Details of the synthesis are described in U.S. Pat. No. 7,462,627, the contents of which are incorporated herein by reference in its entirety.

More specifically, the methods can include:

1) providing one equivalent of 7-ethyl-10-hydroxycamptothecin containing an available 20-hydroxyl group and one or more equivalents of a bifunctinal linker containing an available carboxylic acid group;

2) reacting the two reactants to form a 7-ethyl-10-hydroxycamptothecin-bifunctional linker intermediate in an inert solvent such as dichloromethane (DCM) (or dimethylformamide (DMF), chloroform, toluene or mixtures thereof) in the presence of a coupling reagent such as 1,(3-dimethyl aminopropyl) 3-ethyl carbodiimide (EDC), (or 1,3-diisopropylcarbodiimide (DIPC), any suitable dialkyl carbodiimide, Mukaiyama reagents, (e.g. 2-halo-1-alkyl-pyridinium halides) or propane phosphonic acid cyclic anhydride (PPACA), etc) and a suitable base such as 4-dimethylaminopyridine (DMAP); and

3) reacting one or more equivalents per active site (fore example, 2 equivalents in Example) of the resulting intermediate having an amine group and one equivalent of an activated polymer, such as a PEG-acid in an inert solvent such as dichloromethane (DCM) (or dimethylformamide (DMF), chloroform, toluene or mixtures thereof) in the presence of a coupling reagent such as 1,(3-dimethyl aminopropyl) 3-ethyl carbodiimide (EDC), PPAC (or 1,3-diisopropylcarbodiimide (DIPC), any suitable dialkyl carbodiimide, Mukaiyama reagents, (e.g. 2-halo-1-alkyl-pyridinium halides) or propane phosphonic acid cyclic anhydride (PPACA), etc.), and a suitable base such as 4-dimethylaminopyridine (DMAP), which are available, for example, from commercial sources such as Sigma Chemical, or synthesized using known techniques, at a temperature from 0° C. up to 22° C.

In one preferred aspect, the 10-hydroxyl group of 7-ethyl-10-hydroxycamptothecin is protected prior to step 1).

Protecting groups for the aromatic OH on10-hydroxyl group in 7-ethyl-10-hydroxycamptothecin are preferred because the protected 7-ethyl-10-hydroxycamptothecin intermediates thereof have better solubility and can be purified in highly pure form efficiently and effectively. For example, silyl-containing protecting groups such as TBDPSCl (t-butyldiphenylsilyl chloride), TBDMSCl (t-butyldimethylsilyl chloride) and TMSCl (trimethylsilyl chloride) can be used to protect the 10-hydroxyl group in 7-ethyl-10-hydroxycamptothecin.

The activated polymer, i.e., a polymer containing 1-4 terminal carboxyl acid groups can be prepared, for example, by converting NOF Sunbright-type having terminal OH groups into the corresponding carboxyl acid derivatives using standard techniques well known to those of ordinary skill. See, for example, Examples 1-2 herein as well as commonly assigned U.S. Pat. No. 5,605,976, the contents of which are incorporated herein by reference.

The first and second coupling agents can be the same or different.

Examples of preferred bifunctional linker groups include glycine, alanine, methionine, sarcosine, etc. and syntheses are described in the Examples. Alternative syntheses can be used without undue experimentation.

According to the present invention, the compounds administered include:

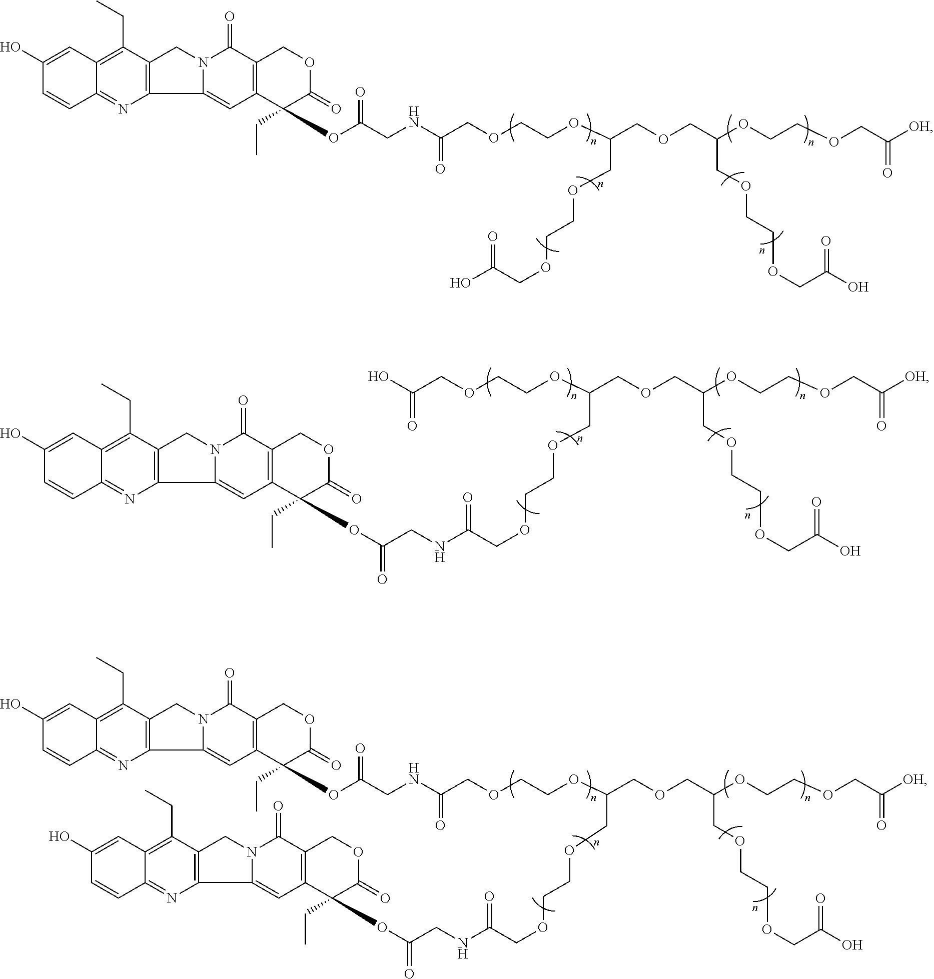

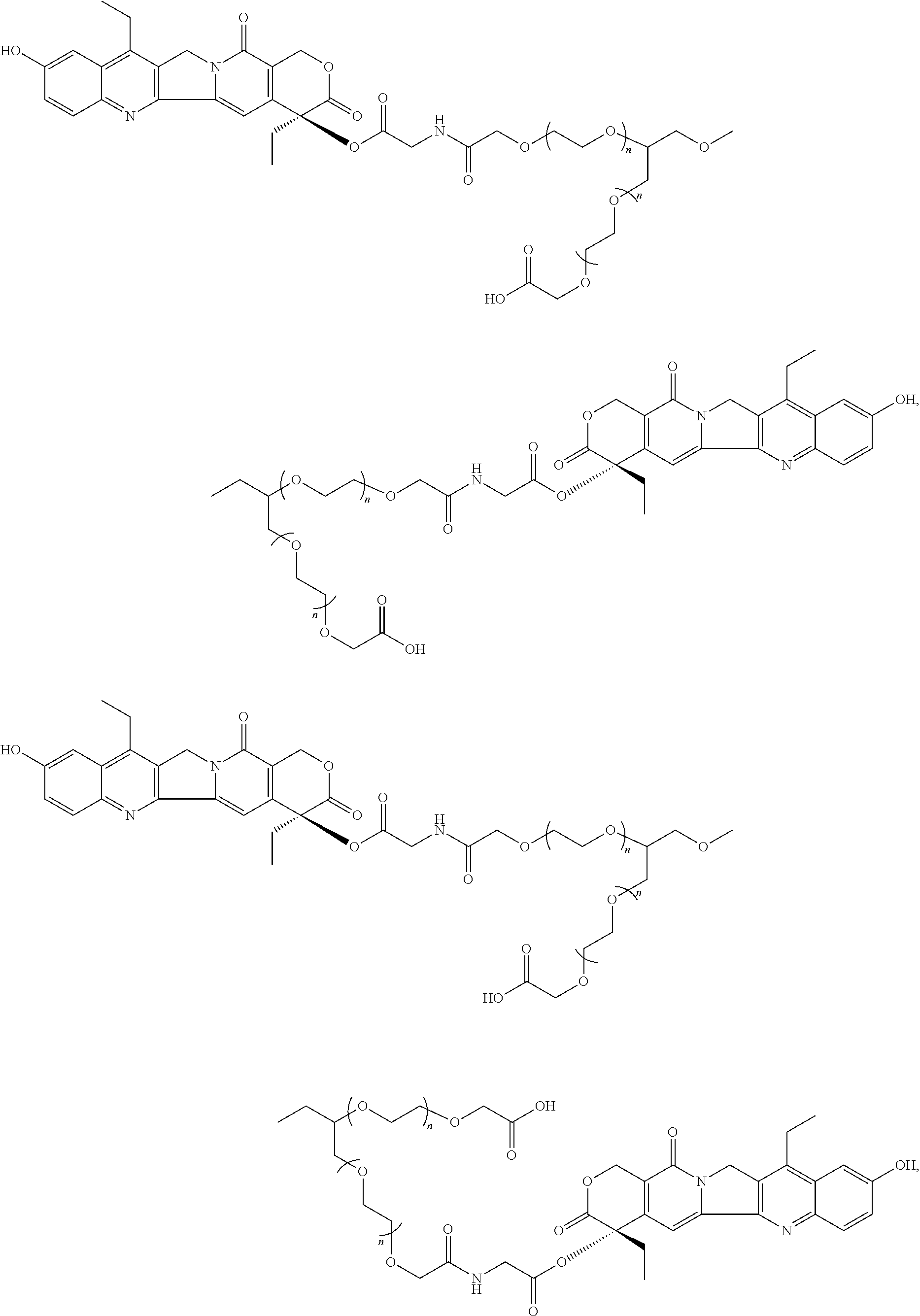

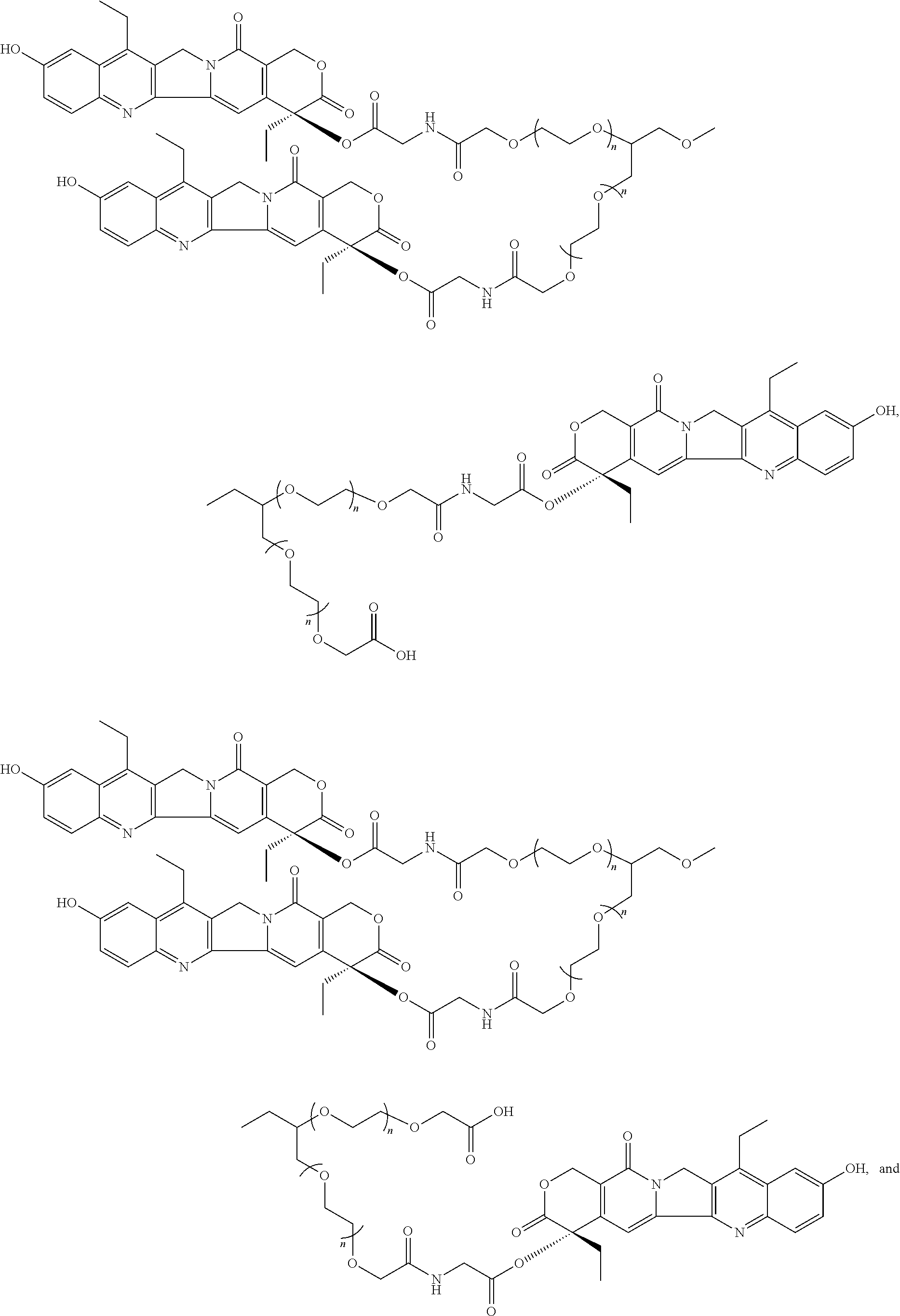

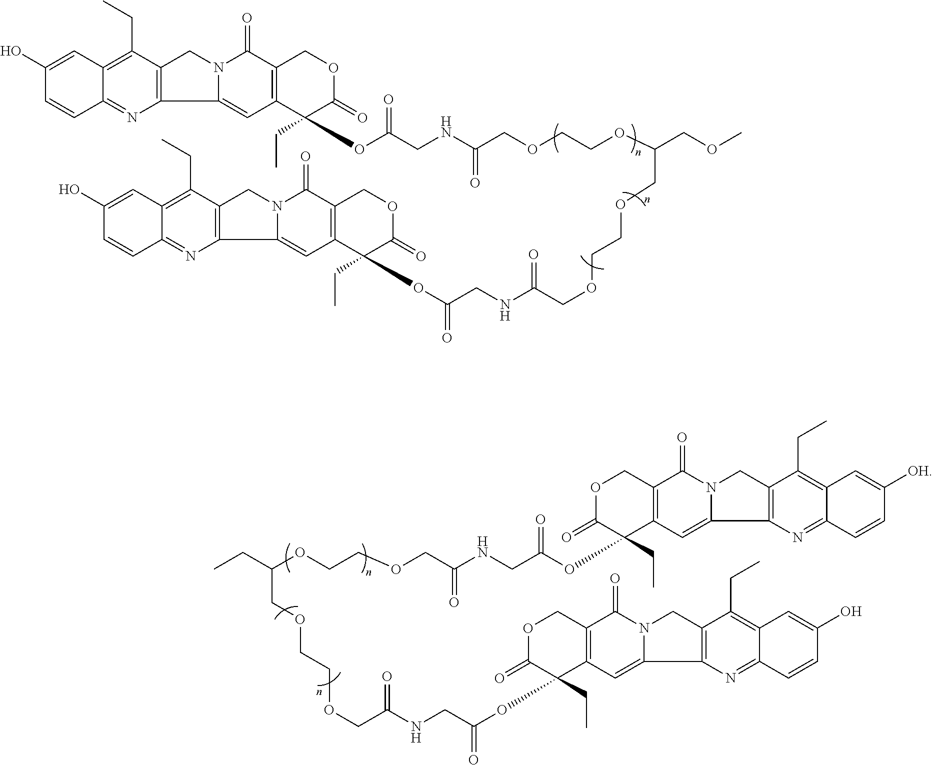

One particularly preferred embodiment includes administering a compound having the structure

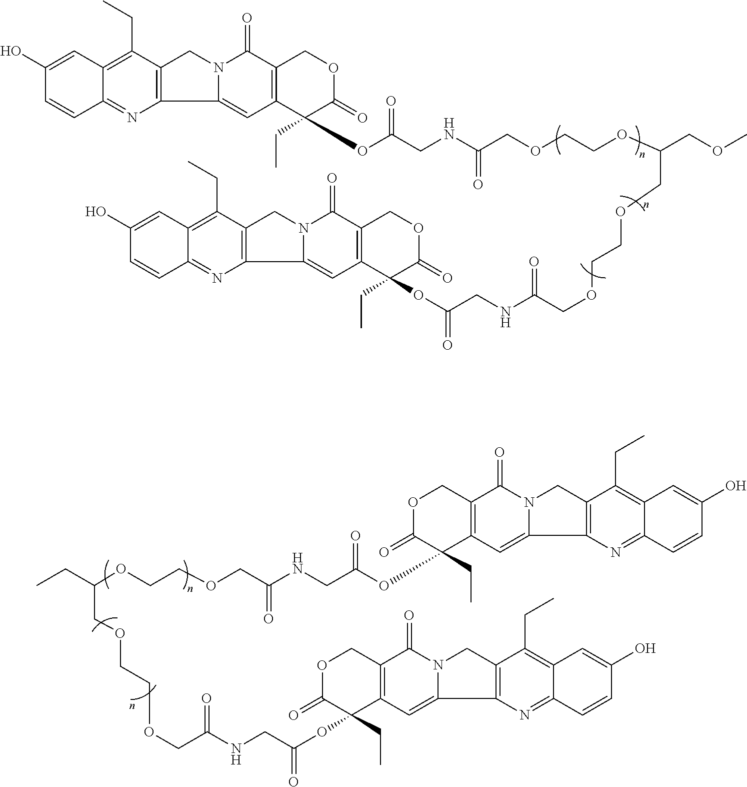

wherein all four arms of the polymer are conjugated to 7-ethyl-10-hydroxycamptothecin through glycine and the polymer portion has the total number average molecular weight of about 40,000 daltons.
C. Combination Therapy with Antisense HIF-1α Oligonucleotide

In a further aspect of the present invention, the methods described herein can be conducted wherein the compound of Formula (I) is administered with a second therapeutic agent for additive effect. The second therapeutic agent includes pharmaceutically active compounds (small molecules with molecular weight less than 1500 daltons, i.e. less than 1000 daltons), antibodies and oligonucleotides. The second therapeutic agent can be administered concurrently or sequentially.

In one aspect, the present invention is conducted wherein the second therapeutic agent is an oligonucleotide which targets pro-angiogenesis pathway genes.

In one preferred aspect, the methods described herein are conducted wherein the compound of Formula (I) is administered with an antisense HIF-1α oligonucleotide. The antisense HIF-1α oligonucleotide used in the method described herein is involved in downregulating the HIF-1α gene or protein expression. HIF-1α gene or protein is associated with angiogenesis or apoptosis. HIF-1α gene/protein is also associated with tumor cells and/or the resistance of tumor cells to anticancer therapeutics.

Hypoxia-inducible factor 1 (HIF-1) is an important regulator of the transcriptional response of mammalian cells to oxygen deprivation. It plays an important role in expression of many genes that control angiogenesis, glucose metabolism, cell proliferation, cell survival, and metastasis in response to hypoxia. Elevated expression of alpha subunit of HIF-1 (HIF-1α) is associated with poor prognosis in many types of solid tumors such as lung, breast, colorectal, brain, pancreatic, ovarian, renal, and bladder cancers. Recently, it has been suggested that HIF and the thioredoxin family are abnormally activated in lymphoma. HIF is frequently activated in lymphoma and it may contribute to disease progression. In one study, 44% of DLBCL (diffuse large B-cell lymphoma) versus 11% of FL (follicular lymphoma) biopsies had moderate-to-high expression of both HIF-1α and HIF-2α. (Evens et al. BJH 2008, 141:676). Trx-1 is frequently overexpressed in many human cancers and its expression has been associated with increased levels of HIF-1α protein and HIF-1α target genes (Welsh et al Mol Cancer therapy).

In one embodiment, the antisense HIF-1α oligonucleotide includes nucleic acids complementary to at least 8 consecutive nucleotides of HIF-1α pre-mRNA or mRNA.

An “oligonucleotide” is generally a relatively short polynucleotide, e.g., ranging in size from about 2 to about 200 nucleotides, or preferably from about 8 to about 50 nucleotides, or more preferably from about 8 to about 30 nucleotides. The oligonucleotides according to the invention are generally synthetic nucleic acids, and are single stranded, unless otherwise specified. The terms, “polynucleotide” and “polynucleic acid” may also be used synonymously herein.

The oligonucleotides (analogs) are not limited to a single species of oligonucleotide but, instead, are designed to work with a wide variety of such moieties. The nucleic acids molecules contemplated can include a phosphorothioate internucleotide linkage modification, sugar modification, nucleic acid base modification and/or phosphate backbone modification. The oligonucleotides can contain natural phosphorodiester backbone or phosphorothioate backbone or any other modified backbone analogues such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

Modifications to the oligonucleotides contemplated by the invention include, for example, the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5′ cap structure

For purposes of the present invention, “cap structure” shall be understood to mean chemical modifications, which have been incorporated at either terminus of the oligonucleotide. The cap can be present at the 5′-terminus (5′-cap) or at the 3′-terminus (3′-cap) or can be present on both termini. A non-limiting example of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. Details are described in WO 97/26270, incorporated by reference herein. The 3′-cap can include for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer, 1993, Tetrahedron 49, 1925; the contents of which are incorporated by reference herein.

A non-limiting list of nucleoside analogs have the structure:

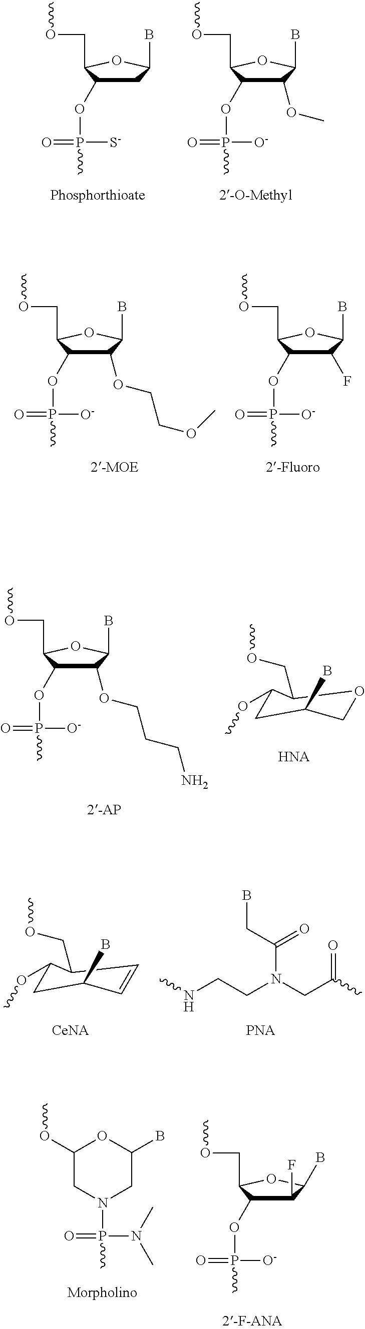

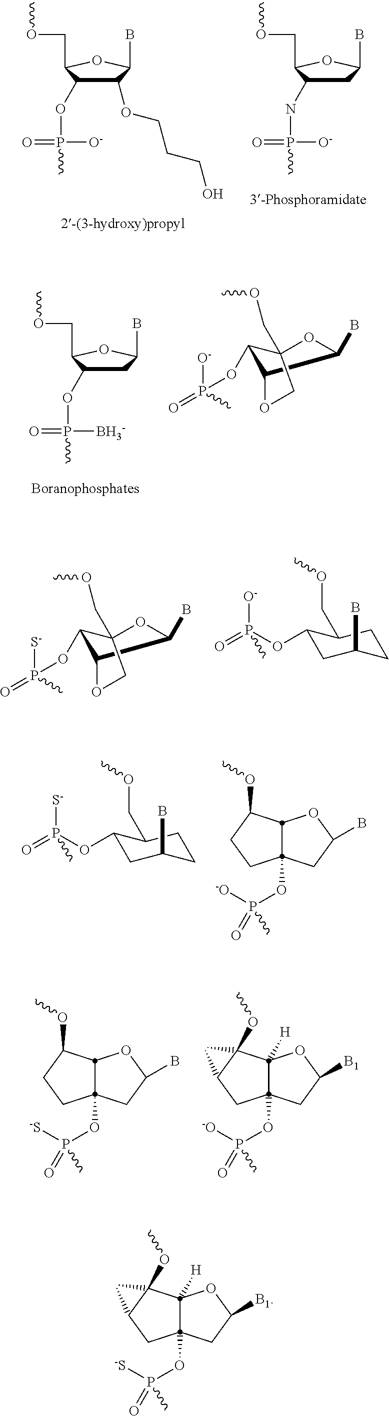

See more examples of nucleoside analogues described in Freier & Altmann; Nucl. Acid Res., 1997, 25, 4429-4443 and Uhlmann; Curr. Opinion in Drug Development, 2000, 3(2), 293-213, the contents of each of which are incorporated herein by reference.

The term “antisense,” as used herein, refers to nucleotide sequences which are complementary to a specific DNA or RNA sequence that encodes a gene product or that encodes a control sequence. The term “antisense strand” is used in reference to a nucleic acid strand that is complementary to the “sense” strand. In the normal operation of cellular metabolism, the sense strand of a DNA molecule is the strand that encodes polypeptides and/or other gene products. The antisense strand serves as a template for synthesis of a messenger RNA (“mRNA”) transcript (a sense strand) which, in turn, directs synthesis of any encoded gene product. Antisense nucleic acid molecules may be produced by any art-known methods, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription or translation. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another nucleic acid sequence. A percent complementarity indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 90%, and 100% complementary. “Perfectly complementary” means that all the contiguous residues of a nucleic acid sequence form hydrogen bonds with the same number of contiguous residues in a second nucleic acid sequence.

The oligonucleotides or oligonucleotide derivatives useful in the method described herein can include from about 10 to about 1000 nucleic acids, and preferably relatively short polynucleotides, e.g., ranging in size from about 8 to about 30 nucleotides in length (e.g., about 8, 9, 10, 11, 12, 13, 14, 15, 16, 17, 18, 19, 20, 21, 22 23, 24, 25, 26, 27, 28, 29, or 30).

In one aspect of useful nucleic acids used in the method described herein, oligonucleotides and oligodeoxynucleotides with natural phosphorodiester backbone or phosphorothioate backbone or any other modified backbone analogues include;

LNA (Locked Nucleic Acid);

PNA (nucleic acid with peptide backbone);

short interfering RNA (siRNA);

microRNA (miRNA);

nucleic acid with peptide backbone (PNA);

phosphorodiamidate morpholino oligonucleotides (PMO);

tricyclo-DNA;

decoy ODN (double stranded oligonucleotide);

catalytic RNA sequence (RNAi);

ribozymes;

aptamers;

spiegelmers (L-conformational oligonucleotides);

CpG oligomers, and the like, such as those disclosed at:

Tides 2002, Oligonucleotide and Peptide Technology Conferences, May 6-8, 2002, Las Vegas, Nev. and Oligonucleotide & Peptide Technologies, 18 & 19 Nov. 2003, Hamburg, Germany, the contents of which are incorporated herein by reference.

In another aspect of the nucleic acids used in the method described herein, oligonucleotides can optionally include any suitable art-known nucleotide analogs and derivatives, including those listed by Table 1, below:

TABLE 1
Representative Nucleotide Analogs And Derivatives

| | |
|---|---|
| 4-acetylcytidine | 5-methoxyaminomethyl-2-thiouridine |
| 5-(carboxyhydroxymethyl)uridine | beta, D-mannosylqueuosine |
| 2′-O-methylcytidine | 5-methoxycarbonylmethyl-2- |
| | thiouridine |
| 5-methoxycarbonylmethyluridine | 5-carboxymethylaminomethyl-2- |
| | thiouridine |
| 5-methoxyuridine | 5-carboxymethylaminomethyluridine |
| Dihydrouridine | 2-methylthio-N6- |
| | isopentenyladenosine |
| 2′-O-methylpseudouridine | N-[(9-beta-D-ribofuranosyl-2- |
| | methylthiopurine-6- |
| | yl)carbamoyl]threonine |
| D-galactosylqueuosine | N-[(9-beta-D-ribofuranosylpurine- |
| | 6-yl)N-methylcarbamoyl]threonine |
| 2′-O-methylguanosine | uridine-5-oxyacetic acid-methylester |
| 2′-halo-adenosine | 2′-halo-cytidine |
| 2′-halo-guanosine | 2′-halo-thymine |
| 2′-halo-uridine | 2′-halo-methylcytidine |
| 2′-amino-adenosine | 2′-amino-cytidine |
| 2′-amino-guanosine | 2′-amino-thymine |
| 2′-amino-uridine | 2′-amino-methylcytidine |
| Inosine | uridine-5-oxyacetic acid |
| N6-isopentenyladenosine | Wybutoxosine |
| 1-methyladenosine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-[(9-beta-D-ribofuranosylpurine- |
| | 6-yl)-carbamoyl]threonine |
| 5-methylcytidine | 2′-O-methyl-5-methyluridine |
| N6-methyladenosine | 2′-O-methyluridine |
| 7-methylguanosine | Wybutosine |
| 5-methylaminomethyluridine | 3-(3-amino-3-carboxy-propyl)uridine |
| Locked-adenosine | Locked-cytidine |
| Locked-guanosine | Locked-thymine |
| Locked-uridine | Locked-methylcytidine |

In one preferred embodiment, the antisense HIF-1α oligonucleotide includes nucleotides that are complementary to at least 8 consecutive nucleotides of the sequence set forth in SEQ ID NO: 1.

Preferably, the oligonucleotides according to the invention described herein include one or more phosphorothioate internucleotide linkages (backbone) and one or more locked nucleic acids (LNA).

One particular embodiment contemplated includes an antisense HIF-1α LNA (SEQ ID NO: 2):

5′-TGGcaagcatccTGTa-3′

where the upper case letter represents LNA and internucleoside linkage is phosphorothioate; and
LNA includes 2′-O, 4′-C methylene bicyclonucleotide as shown below:

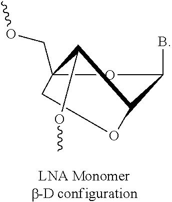

See, for example, the detailed description of HIF-1α LNA disclosed in U.S. Patent Application Publication Nos. 2004/0096848, entitled “Oligomeric Compounds for the Modulation HIF-1 Alpha Expression” and 2006/0252721, entitled “Potent LNA Oligonucleotides for Inhibition of HIF-1α Expression”, the contents of each of which are incorporated herein by reference in its entirety. See also WO2008/113832, the contents of which are incorporated herein by reference in its entirety.

In a further aspect, the present invention is contemplated to include oligonucleotides which target, for example, but are not limited to, oncogenes, pro-cell proliferation pathway genes, viral infectious agent genes, and pro-inflammatory pathway genes. A non-limiting list of therapeutic oligonucleotides includes antisense survivin oligonucleotides, antisense ErbB3 oligonucleotides, antisense β-catenin oligonucleotides, antisense androgen receptor oligonucleotides, antisense PIK3CA oligonucleotides, antisense HSP27 oligonuucleotides, anstisense Gli 2 oligonucleotides, and antisense Bcl-2 oligonucleotides. Additional examples of suitable target genes are described in WO 03/74654, PCT/US03/05028, WO2008/138904, WO2008/132234, WO 2009/068033, WO2009/071082, WO 2010/001349, WO 2010/007522, and U.S. patent application Ser. No. 10/923,536, the contents of which are incorporated by reference herein.

### D. Compositions/Formulations

Pharmaceutical compositions containing the polymer conjugates described herein may be manufactured by processes well known in the art, e.g., using a variety of well-known mixing, dissolving, granulating, levigating, emulsifying, encapsulating, entrapping or lyophilizing processes. The compositions may be formulated in conjunction with one or more physiologically acceptable carriers comprising excipients and auxiliaries which facilitate processing of the active compounds into preparations which can be used pharmaceutically. Proper formulation is dependent upon the route of administration chosen. Parenteral routes are preferred in many aspects of the invention.

For injection, including, without limitation, intravenous, intramusclular and subcutaneous injection, the compounds of Formula (I) described herein may be formulated in aqueous solutions, preferably in physiologically compatible buffers such as physiological saline buffer or polar solvents including, without limitation, a pyrrolidone or dimethylsulfoxide.

The compounds described herein may also be formulated for parenteral administration, e.g., by bolus injection or continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form, such as, without limitation, a salt (preferred) of the active compound. Additionally, suspensions of the active compounds may be prepared in a lipophilic vehicle. Suitable lipophilic vehicles include fatty oils such as sesame oil, synthetic fatty acid esters such as ethyl oleate and triglycerides, or materials such as liposomes. Aqueous injection suspensions may contain substances that increase the viscosity of the suspension, such as sodium carboxymethyl cellulose, sorbitol, or dextran. Optionally, the suspension may also contain suitable stabilizers and/or agents that increase the solubility of the compounds to allow for the preparation of highly concentrated solutions. Alternatively, the active ingredient may be in powder form for constitution with a suitable vehicle, e.g., sterile, pyrogen-free water, before use.

For oral administration, the compounds can be formulated by combining the active compounds with pharmaceutically acceptable carriers well-known in the art. Such carriers enable the compounds of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropyl-methylcellulose, sodium carboxy-methylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the compounds of the present invention can conveniently be delivered in the form of an aerosol spray using a pressurized pack or a nebulizer and a suitable propellant.

The compounds may also be formulated in rectal compositions such as suppositories or retention enemas, using, e.g., conventional suppository bases such as cocoa butter or other glycerides.

In addition to the formulations described previously, the compounds may also be formulated as depot preparations. Such long acting formulations may be administered by implantation (for example, subcutaneously or intramuscularly) or by intramuscular injection. A compound of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Other delivery systems such as liposomes and emulsions can also be used.

Additionally, the compounds may be delivered using a sustained-release system, such as semi-permeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the particular compound, additional stabilization strategies may be employed.

### E. Dosages

A therapeutically effective amount refers to an amount of a compound effective to inhibit, prevent, alleviate or ameliorate a pathological condition such as angiogenesis or angiogenesis-associated condition. Determination of a therapeutically effective amount is well within the capability of those skilled in the art, especially in light of the disclosure herein.

For any compound used in the methods of the present invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the composition, e.g., used as a prodrug, that is administered will depend upon the parent molecule included therein (in this case, 7-ethyl-10-hydroxy-camptothecin). Generally, the amount of prodrug used in the methods described herein is that amount which effectively achieves the desired therapeutic result in mammals. Naturally, the dosages of the various prodrug compounds can vary somewhat depending upon the parent compound, rate of in vivo hydrolysis, molecular weight of the polymer, etc. In addition, the dosage, of course, can vary depending upon the dosage form and route of administration.

In general, however, the polymeric ester derivatives of 7-ethyl-10-hydroxy-camptothecin described herein can be administered in amounts ranging from about 0.3 to about 90 mg/m² body surface, and preferably from about 0.5 to about 50 mg/m² body surface/dose, yet preferably from about 1 to about 18 mg/m² body surface/dose, and even more preferably from about 1.25 mg/m² body surface/dose to about 16.5 mg/m² body surface/dose for systemic delivery. Some particular doses include one of the following: 1.25, 2.5, 5, 9, 10, 12, 13, 14, 15, 16 and 16.5 mg/m²/dose. One preferred dosage includes 5 mg/m² body surface/dose. In this aspect, the amount is the weight of 7-ethyl-10-hydroxycamptothecin included in the compound of Formula (I).

The compounds can be administered in amounts ranging from about 0.3 to about 90 mg/m² body surface/week such as, for example, from about 1 to about 18 mg/m² body surface/week. In particular embodiments, the dose regimens can be, for example, from about 5 to about 7 mg/m² body surface weekly for 3 weeks in 4-week cycles, from about 1.25 to about 45 mg/m² one injection every 3 weeks, and/or from about 1 to about 16 mg/m² three injections weekly in a four week cycle.

The treatment protocol can be based, for example, on a single dose administered once every three weeks or divided into multiple doses which are given as part of a multi-week treatment protocol. Thus, the treatment regimens can include, e.g., one dose every three weeks for each treatment cycle and, alternatively one dose weekly for three weeks followed by one week off for each cycle. It is also contemplated that the treatment will be given for one or more cycles until the desired clinical result is obtained.

The range set forth above is illustrative and those skilled in the art will determine the optimal dosing of the prodrug selected based on clinical experience and the treatment indication. Moreover, the exact formulation, route of administration and dosage can be selected by the individual physician in view of the patient's condition. The precise dose will depend on the stage and severity of the condition, and the individual characteristics of the patient being treated, as will be appreciated by one of ordinary skill in the art.

Additionally, toxicity and therapeutic efficacy of the compounds described herein can be determined by standard pharmaceutical procedures in cell cultures or experimental animals using methods well-known in the art.

In some preferred embodiments, the treatment protocol includes administering the amount ranging from about 1.25 to about 16.5 mg/m² body surface/dose weekly for three weeks, followed by one week without treatment and repeating for about 3 cycles or more until the desired results are observed. The amount administered per each cycle can range from about 2.5 to about 16.5 mg/m² body surface/dose.

In one particular embodiment, the polymeric ester derivatives of 7-ethyl-10-hydroxycamptothecin can be administered in one dose, such as 5, 9 or 10 mg/m² weekly for three weeks, followed by one week without treatment. The dosage of the treatment cycle can be designed as an escalating dose regimen when two or more treatment cycles are applied. The polymeric drug is preferably administered via IV infusion.

In another particular embodiments, the compound of Formula (I) is administered in a dose from about 12 to about 16 mg/m² body surface/dose. The dose can be given weekly. The treatment protocol includes administering the compound of Formula (I) in amounts ranging from about 12 to about 16 mg/m² body surface/dose weekly for three weeks, followed by one week without treatment.

In yet another particular embodiment, the dose regiment can be about 10 mg/m² body surface/dose every three weeks.

Alternative embodiments include: for the treatment of pediatric patients, a regimen based on a protocol of about 1.85 mg/m² body surface/dose daily for 5 days every three weeks, a protocol of from about 1.85 to about 7.5 mg/m² body surface/dose daily for 3 days every 25 days, or a protocol of about 22.5 mg/m² body surface/dose once every three weeks, and for the treatment of adult patients, a protocol based on about 13 mg/m² body surface/dose every three weeks or about 4.5 mg/m² body surface/dose weekly for four weeks every six weeks. The compounds described herein can be administered in combination with a second therapeutic agent. In one embodiment, the combination therapy includes a protocol of about 0.75 mg/m² body surface/dose daily for 5 days each cycle in combination with a second agent.

Alternatively, the compounds can be administered based on body weight. The dosage range for systemic delivery of a compound of Formula (I) in a mammal will be from about 1 to about 100 mg/kg/week and is preferably from about 2 to about 60 mg/kg/week. Thus, the amounts can range from about 0.1 mg/kg body weight/dose to about 30 mg/kg body weight/dose, preferably, from about 0.3 mg/kg to about 10 mg/kg. Specific doses such as 10 mg/kg at q2d×5 regimen (multiple dose) or 30 mg/kg on a single dose regimen can be administered.

In all aspects of the invention where polymeric conjugates are administered, the dosage amount mentioned is based on the amount of 7-ethyl-10-hydroxycamptothecin rather than the amount of polymeric conjugate administered. The actual weight of the PEG-conjugated 7-ethyl-10-hydroxycamptothecin will vary depending on the weight of PEG and the loading of the PEG (e.g., optionally from one to four equivalents of 7-ethyl-10-hydroxycamptothecin per multi-arm PEG). It is contemplated that the treatment will be given for one or more cycles until the desired clinical result is obtained. The exact amount, frequency and period of administration of the compound of the present invention will vary, of course, depending upon the sex, age and medical condition of the patient as well as the severity of the disease as determined by the attending clinician.

Further aspects of the present invention include combining the compounds described herein with other therapies such as a second therapeutic agent or radiotherapy for synergistic or additive benefit.

The combination therapy protocol includes administering an antisense oligonucleotide in an amount of from about 2 to about 100 mg/kg/dose (e.g., 2, 3, 4, 5, 6, 8, 10, 15, 20, 25, 30, 35, 40, 50, 60, 70, 100 mg/kg/dose). For example, the combination therapy regimen dose includes treatment with an antisense HIF-1α oligonucleotide in an amount of from about 2 to about 50 mg/kg/dose. Preferably, the antisense oligonucleotide administered in the combination therapy is in an amount of from about 3 to about 25 mg/kg/dose.

In one aspect of the combination therapy, the protocol includes administering an antisense HIF-1α oligonucleotide in an amount of about 4 to about 18 mg/kg/dose weekly, or about 4 to about 9.5 mg/kg/dose weekly.

In one particular embodiment, the combination therapy protocol includes an antisense HIF-1α oligonucleotide in an amount of about 4 to about 18 mg/kg/dose weekly for 3 weeks in a six week cycle (i.e. about 8 mg/kg/dose). Another particular embodiment includes about 4 to about 9.5 mg/kg/dose weekly (i.e., about 4 mg/kg/dose).

### EXAMPLES

The following examples serve to provide further appreciation of the invention but are not meant in any way to restrict the effective scope of the invention. The bold-faced numbers, e.g., compound numbers, recited in the Examples correspond to those shown in the figures.

General Procedures. All reactions were run under an atmosphere of dry nitrogen or argon. Commercial reagents were used without further purification. All PEG compounds were dried under vacuum or by azeotropic distillation from toluene prior to use. ¹³C NMR spectra were obtained at 75.46 MHz using a Varian Mercury® 300 NMR spectrometer and deuterated chloroform and methanol as the solvents unless otherwise specified. Chemical shifts (δ) are reported in parts per million (ppm) downfield from tetramethylsilane (TMS).

HPLC Method. The reaction mixtures and the purity of intermediates and final products were monitored by a Beckman Coulter System Gold® HPLC instrument. It employs a ZOBAX° 300SB C8 reversed phase column (150×4.6 mm) or a Phenomenex Jupiter® 300A C18 reversed phase column (150×4.6 mm) with a multiwavelength UV detector, using a gradient of 10-90% of acetonitrile in 0.05% trifluoroacetic acid (TFA) at a flow rate of 1 mL/min.)

### Example 1

### ^{40k}4arm-PEG-tBu Ester (Compound 2)

^{40k}4arm-PEG-OH (12.5 g, 1 eq.) was azeotroped with 220 mL of toluene to remove 35 mL of toluene/water. The solution was cooled to 30° C. and 1.0 M potassium t-butoxide in t-butanol (3.75 mL, 3 eq×4=12 eq.) was added. The mixture was stirred at 30° C. for 30 min and then t-butyl bromoacetate (0.975 g, 4 eq.×4=16 eq.) was added. The reaction was kept at 30° C. for 1 hour and then was cooled to 25° C. 150 mL of ether was slowly added to precipitate product. The resulting suspension was cooled to 17° C. and stayed at 17° C. for half hour. The crude product was filtered and the wet cake was washed with ether twice (2×125 mL). The isolated wet cake was dissolved in 50 ml of DCM and the product was precipitated with 350 ml of ether and filtered. The wet cake was washed with ether twice (2×125 mL). The product was dried under vacuum at 40° C. (yield=98%, 12.25 g). ¹³C NMR (75.4 MHz, CDCl₃): δ 27.71, 68.48-70.71 (PEG), 80.94, 168.97.

### Example 2

### ^{40k}4arm-PEG Acid (Compound 3)

^{40k}4arm-PEG-tBu ester (compound 2, 12 g) was dissolved in 120 mL of DCM and then 60 mL of TFA were added. The mixture was stirred at room temperature for 3 hours and then the solvent was removed under vacuum at 35° C. The resulting oil residue was dissolved in 37.5 mL of DCM. The crude product was precipitated with 375 mL of ether. The wet cake was dissolved in 30 mL of 0.5% NaHCO₃. The product was extracted with DCM twice (2×150 ml). The combined organic layers were dried over 2.5 g of MgSO₄. The solvent was removed under vacuum at room temperature. The resulting residue was dissolved in 37.5 mL of DCM and the product was precipitated with 300 mL of ether and filtered. The wet cake was washed with ether twice (2×125 ml). The product was dried under vacuum at 40° C. (yield=90%, 10.75 g). ¹³C NMR (75.4 MHz, CDCl₃): δ 67.93-71.6 (PEG), 170.83.

### Example 3

### TBDPS-(10)-(7-ethyl-10-hydroxycamptothecin) (Compound 5)

To a suspension of 7-ethyl-10-hydroxycamptothecin (compound 4, 2.0 g, 5.10 mmol, 1 eq.) in 100 mL of anhydrous DCM were added Et₃N (4.3 mL, 30.58 mmol, 6 eq.) and TBDPSCl (7.8 mL, 30.58 mmol, 6 eq.). The reaction mixture was heated to reflux overnight and then, was washed with a 0.2 N HCl solution (2×50 mL), a saturated NaHCO₃ solution (100 mL) and brine (100 mL). The organic layer was dried over MgSO₄, filtered and evaporated under vacuum. The residue was dissolved in anhydrous DCM and precipitated by addition of hexanes. The precipitation with DCM/hexanes was repeated to get rid of excess TBDPSCl. The solids were filtered and dried under vacuum to give 2.09 g of product. (65% yield). ¹H NMR (300 MHz, CDCl₃): δ 0.90 (3H, t, J=7.6 Hz), 1.01 (3H, t, J=7.3 Hz), 1.17 (9H, s), 1.83-1.92 (2H, m), 2.64 (2H, q, 6.9 Hz), 3.89 (1H, s, OH), 5.11 (2H, s), 5.27 (1H, d, J=16.1 Hz), 5.72 (1H, d, J=16.4 Hz), 7.07 (2H, d, J=2.63 Hz), 7.36-7.49 (7H, m), 7.58 (1H, s), 7.75-7.79 (4H, m), 8.05 (1H, d, J=9.4 Hz). ¹³C NMR (75.4 MHz, CDCl₃): δ 7.82, 13.28, 19.52, 22.86, 26.48, 31.52, 49.23, 66.25, 72.69, 97.25, 110.09, 117.57, 125.67, 126.57, 127.65, 127.81, 130.02, 131.69, 131.97, 135.26, 143.51, 145.05, 147.12, 149.55, 149.92, 154.73, 157.43, 173.72.

### Example 4

### TBDPS-(10)-(7-ethyl-10-hydroxycamptothecin)-(20)-Gly-Boc (Compound 6)

To a 0° C. solution of TBDPS-(10)-(7-ethyl-10-hydroxycamptothecin) (compound 5, 3.78 g, 5.99 mmol, 1 eq.) and Boc-Gly-OH (1.57 g, 8.99 mmol, 1.5 eq.) in 100 mL of anhydrous DCM was added EDC (1.72 g, 8.99 mmol, 1.5 eq.) and DMAP (329 mg, 2.69 mmol, 0.45 eq.). The reaction mixture was stirred at 0° C. until HPLC showed complete disappearance of the starting material (approx. 1 hour and 45 minutes). The organic layer was washed with a 0.5% NaHCO₃ solution (2×50 mL), water (1×50 mL), a 0.1 N HCl solution (2×50 mL) and brine (1×50 mL); and dried over MgSO₄. After filtration and evaporation under vacuum, 4.94 g of crude product were obtained (quantitative yield). The crude solid was used in the next reaction without further purification. ¹H NMR (300 MHz, CDCl₃): δ 0.89 (3 H, t, J=7.6 Hz), 0.96 (3H, t, J=7.5 Hz), 1.18 (9H, s), 1.40 (9H, s), 2.07-2.29 (3H, m), 2.64 (2H, q, 7.5 Hz), 4.01-4.22 (2H, m), 5.00 (1H, br s), 5.01 (2H, s), 5.37 (1H, d, J=17.0 Hz), 5.66 (1H, d, J=17.0 Hz), 7.08 (1H, d, J=2.34 Hz), 7.16 (1H, s), 7.37-7.50 (7H, m), 7.77 (4H, d, J=7.6 Hz), 8.05 (1H, d, J=9.4 Hz). ¹³C NMR (75.4 MHz, CDCl₃): δ 7.52, 13.30, 19.50, 22.86, 26.45, 28.21, 31.64, 42.28, 49.14, 67.00, 76.65, 79.96, 95.31, 110.13, 118.98, 125.75, 126.45, 127.68, 127.81, 130.03, 131.54, 131.92, 135.25, 143.65, 144.91, 145.19, 147.08, 149.27, 154.75, 155.14, 157.10, 166.98, 169.17.

### Example 5

### TBDPS-(10)-(7-ethyl-10-hydroxycamptothecin)-(20)-Gly.HCl (Compound 7)

To a solution of TBDPS-(10)-(7-ethyl-10-hydroxycamptothecin)-(20)-Gly-Boc (compound 6, 1 g, 1.27 mmol) in 5 mL anhydrous dioxane was added 5 mL of a 4 M solution of HCl in dioxane. The reaction mixture was stirred at room temperature until HPLC showed complete disappearance of the starting material (1 hour). The reaction mixture was added to 50 mL of ethyl ether and the resulting solid was filtered. The solid was dissolved in 50 mL DCM and washed with brine (pH was adjusted to 2.5 by addition of a saturated NaHCO₃ solution). The organic layer was dried over MgSO₄, filtered and evaporated under vacuum. The residue was dissolved in 5 mL of DCM and precipitated by addition of 50 mL ethyl ether. Filtration afforded 770 mg (84% yield) final product. ¹H NMR (300 MHz, CDCl₃): δ 0.84 (3H, t, J=7.6 Hz), 1.05 (3H, t, J=7.3 Hz), 1.16 (9H, s), 2.15-2.30 (3H, m), 2.59 (2H, q, 7.6 Hz), 4.16 (1H, d, J=17.9 Hz), 4.26 (1H, d, J=17.9 Hz), 5.13 (2H, s), 5.46 (1H, d, J=17.0 Hz), 5.60 (1H, d, J=17.0 Hz), 7.11 (1H, d, J=2.34 Hz), 7.30 (1H, s), 7.40-7.51 (6H, m), 7.56 (1H, dd, J=2.34, 9.4 Hz), 7.77 (4H, dd, J=7.6, 1.6 Hz), 7.98 (1H, d, J=9.1 Hz). ¹³C NMR (75.4 MHz, CDCl₃): δ 8.09, 13.72, 20.26, 23.61, 26.94, 31.83, 41.01, 50.71, 67.62, 79.51, 97.03, 111.65, 119.69, 127.13, 128.97, 128.99, 129.11, 131.43, 131.96, 133.00, 133.03, 136.51, 145.62, 145.81, 147.24, 148.29, 150.58, 156.27, 158.68, 167.81, 168.34.

### Example 6

### ^{40k}4arm-PEG-Gly-(20)-(7-ethyl-10-hydroxycamptothecin)-(10)-TBDPS (Compound 8)

To a solution of ^{40k}4arm-PEGCOOH (compound 3, 1.4 g, 0.036 mmol, 1 eq.) in 14 mL of anhydrous DCM was added TBDPS-(10)-(7-ethyl-10-hydroxycamptothecin)-(20)-Gly.HCl (compound 7, 207 mg, 0.29 mmol, 2.0 eq. per active site), DMAP (175 mg, 1.44 mmol, 10 eq.) and PPAC (0.85 mL of a 50% solution in EtOAc, 1.44 mmol, 10 eq.). The reaction mixture was stirred at room temperature overnight and then, evaporated under vacuum. The resulting residue was dissolved in DCM and the product was precipitated with ether and filtered. The residue was recrystallized with DMF/IPA to give the product (1.25 g). ¹³C NMR (75.4 MHz, CDCl₃): δ 7.45, 13.20, 19.39, 22.73, 26.42, 31.67, 40.21, 49.01, 66.83, 95.16, 110.02, 118.83, 125.58, 126.40, 127.53, 127.73, 129.96, 131.49, 131.76, 131.82, 135.12, 143.51, 144.78, 145.13, 146.95, 149.21, 154.61, 156.92, 166.70, 168.46, 170.30.

### Example 7

### ^{40k}4arm-PEG-Gly(20)-(7-ethyl-10-hydroxycamptothecin) (Compound 9)

To compound ^{40k}4arm-PEG-Gly-(20)-(7-ethyl-10-hydroxycamptothecin)-(10)-TBDPS (compound 8, 1.25 g) was added a solution of TBAF (122 mg, 0.46 mmol, 4 eq.) in a 1:1 mixture of THF and a 0.05 M HCl solution (12.5 mL). The reaction mixture was stirred at room temperature for 4 hours and then, extracted with DCM twice. The combined organic phases were dried over MgSO₄, filtered and evaporated under vacuum. The residue was dissolved in 7 mL of DMF and precipitated with 37 mL IPA. The solid was filtered and washed with IPA. The precipitation with DMF/IPA was repeated. Finally the residue was dissolved in 2.5 mL of DCM and precipitated by addition of 25 mL of ether. The solid was filtered and dried at 40° C. in vacuum oven overnight (860 mg). ¹³C NMR (75.4 MHz, CDCl₃): δ 7.48, 13.52, 22.91, 31.67, 40.22, 49.12, 66.95, 94.82, 105.03, 118.68, 122.54, 126.37, 128.20, 131.36, 142.92, 144.20, 144.98, 147.25, 148.29, 156.44, 156.98, 166.82, 168.49, 170.39. This NMR data shows no sign of PEG-COOH which indicates that all of the COOH reacted. The loading, as determined by fluorescence detection was found to be 3.9 which is consistent with full loading of the 7-ethyl-10-hydroxycamptothecin on each of the four branches of the polymer. Repeated runs of this experiments at much larger scale yielded consistent results.

### Example 8

### Boc-(10)-(7-ethyl-10-hydroxycamptothecin) (Compound 10)

To a suspension of 7-ethyl-10-hydroxycamptothecin (compound 4, 2.45 g, 1 eq.) in 250 mL of anhydrous DCM at room temperature under N₂ were added di-tert-butyl dicarbonate (1.764 g, 1.3 eq.) and anhydrous pyridine (15.2 mL, 30 eq.). The suspension was stirred overnight at room temperature. The hazy solution was filtered through celite (10 g) and the filtrate was washed with 0.5 N HCl three times (3×150 mL) and a NaHCO₃ saturated solution (1×150 ml). The solution was dried over MgSO₄ (1.25 g). The solvent was removed under vacuum at 30° C. The product was dried under vacuum at 40° C. (yield=82%, 2.525 g) ¹³C NMR (75.4 MHz, CDCl₃) δ 173.53, 157.38, 151.60, 151.28, 150.02, 149.70, 147.00, 146.50, 145.15, 131.83, 127.19, 127.13, 124.98, 118.53, 113.88, 98.06, 84.26, 72.80, 66.18, 49.33, 31.62, 27.73, 23.17, 13.98, 7.90.

### Example 9

### Boc-(10)-(7-ethyl-10-hydroxycamptothecin)-(20)-Ala-Bsmoc (Compound 11)

To a solution of Boc-(10)-(7-ethyl-10-hydroxycamptothecin) (compound 10, 0.85 g, 1.71 mmol) and Bsmoc-Ala (0.68 g, 2.30 mmol) in anhydrous CH₂Cl₂ (20 mL) were added EDC (0.51 g, 2.67 mmol) and DMAP (0.065 g, 0.53 mmol) at 0° C. The mixture was stirred at 0° C. for 45 min under N₂, then warmed up to room temperature. When completion of the reaction was confirmed by HPLC, the reaction mixture was washed with 1% NaHCO₃ (2×50 ml), H₂O (50 mL) and 0.1 N HCl (2×50 mL). The organic phase was dried with anhydrous MgSO₄ and filtrated. Solvent was removed under reduced pressure. The resulting solid was dried under vacuum below 40° C. overnight to give the product of 1.28 g with the yield of 95%. ¹³C NMR (75.4 MHz, CDCl₃) δ: 171.16, 166.83, 157.16, 154.78, 151.59, 151.33, 149.82, 147.17, 146.68, 145.35, 145.15, 139.08, 136.88, 133.60, 131.83, 130.45, 130.40, 130.33, 127.40, 127.08, 125.32, 125.14, 121.38, 120.01, 114.17, 95.90, 84.38, 77.19, 76.64, 67.10, 56.66, 53.45, 49.96, 49.34, 31.7, 27.76, 17.94, 14.02, 7.53. ESI-MS, 786.20 [M+H]⁺.

### Example 10

### Boc-(10)-(7-ethyl-10-hydroxycamptothecin)-(20)-Ala (Compound 12)

A solution of Boc-(10)-(7-ethyl-10-hydroxycamptothecin)-(20)-Ala-Bsmoc (compound 11, 4.2 g, 5.35 mmol) and 4-piperidinopiperidine (1.17 g, 6.96 mmol) in anhydrous CH₂Cl₂ (200 ml) was stirred at room temperature for 5 hours. This mixture was then washed with 0.1 N HCl (2×40 ml), followed by drying the organic layer over anhydrous MgSO₄. This solution was filtered, and the solvent was removed by vacuum distillation to yield 2.8 g of product with purity of 93%, determined by HPLC. This product was further purified by trituration with ether (3×20 ml), and then trituration with ethyl acetate (4×20 ml) to yield 1.52 g (2.70 mmol) with purity 97%. ¹³C NMR (75.4 MHz, CDCl₃) δ 168.39, 166.63, 156.98, 151.20, 151.15, 149.69, 146.67, 146.56, 145.37, 144.53, 131.66, 127.13, 124.99, 119.80, 113.82, 96.15, 84.21, 77.67, 67.16, 49.48, 49.06, 31.56, 27.74, 23.14, 15.98, 13.98, 7.57.

### Example 11

### ^{40k}4arm-PEG-Ala-(20)-(7-ethyl-10-hydroxycamptothecin)-(10)-Boc (Compound 13)

To anhydrous CH₂Cl₂ (100 mL) Boc-(10)-(7-ethyl-10-hydroxycamptothecin)-(20)-Ala (compound 12, 1.50 g, 2.5 mmol) and 4armPEG-COOH (compound 3, 10.01 g, 1.0 mmol) were added at room temperature. The solution was cooled to 0° C., followed by addition of EDC (0.29 g, 1.5 mmol) and DMAP (0.30 g, 2.5 mmol). The mixture was stirred at 0° C. for 1 hour under N₂. Then it was kept at room temperature overnight. The solvent was evaporated under reduced pressure. The residue was dissolved in 40 mL of DCM, and the crude product was precipitated with ether (300 mL). The wet solid resulting from filtration was dissolved in a mixture of DMF/IPA (60/240 mL) at 65° C. The solution was allowed to cool down to room temperature within 2˜3 hours, and the product was precipitated. Then, the solid was filtered and washed with ether (2×200 mL). The wet cake was dried under vacuum below 40° C. overnight to give product of 8.5 g.

### Example 12

### ^{40k}4arm-PEG-Ala-(20)-(7-ethyl-10-hydroxycamptothecin) (Compound 14)

To a solution (130 mL) of 30% TFA in anhydrous CH₂Cl₂ ^{40k}4arm-PEG-Ala-(20)-(7-ethyl-10-hydroxycamptothecin)-(10)-Boc (compound 13, 7.98 g) was added at room temperature.

The mixture was stirred for 3 hours, or until the disappearance of starting material was confirmed by HPLC. The solvents were removed as much as possible under vacuum at 35° C. The residues were dissolved in 50 mL of DCM, and the crude product was precipitated with ether (350 mL) and filtered. The wet solid was dissolved in a mixture of DMF/IPA (50/200 mL) at 65° C. The solution was allowed to cool down to room temperature within 2˜3 hours, and the product was precipitated. Then the solid was filtered and washed with ether (2×200 mL). The wet cake was dried under vacuum below 40° C. overnight to give product of 6.7 g. ¹³C NMR (75.4 MHz, CDCl₃) δ: 170.75, 169.30, 166.65, 157.00, 156.31, 148.36, 147.19, 145.03, 144.29, 143.00, 131.49, 128.26, 126.42, 122.47, 118.79, 105.10, 94.57, 78.08, 77.81, 77.20, 71.15, 70.88, 70.71, 70.33, 70.28, 70.06, 69.93, 69.57, 66.90, 49.14, 47.14, 31.53, 22.95, 17.78, 13.52, 7.46.

### Example 13

### Boc-(10)-(7-ethyl-10-hydroxycamptothecin)-(20)-Met-Bsmoc (Compound 15)

To a solution of Boc-(10)-7-ethyl-10-hydroxycamptothecin (compound 10, 2.73 g, 5.53 mmol) and Bsmoc-Met (3.19 g, 8.59 mmol) in anhydrous CH₂Cl₂ (50 mL) were added EDC (1.64 g, 8.59 mmol) and DMAP (0.21 g, 1.72 mmol) at 0° C. The mixture was stirred at 0° C. for 45 minutes under N₂, then warmed up to room temperature. When completion of the reaction was confirmed by HPLC, the reaction mixture was washed with 1% NaHCO₃ (2×100 ml), H₂O (100 mL) and 0.1 N HCl (2×100 mL). The organic phase was dried with anhydrous MgSO₄ and filtrated. Solvents were removed under reduced pressure. The resulting solid was dried under vacuum below 40° C. overnight to give the product of 4.2 g with the yield of 88%. ¹³C NMR (75.4 MHz, CDCl₃) δ: 170.3, 166.8, 157.1, 155.2, 151.4, 151.2, 149.7, 147.0, 146.6, 145.3, 145.1, 138.9, 136.6, 133.5, 131.7, 130.5, 130.3, 130.2, 127.3, 127.0, 125.3, 125.1, 121.2, 119.8, 114.1, 96.1, 84.3, 76.7, 67.0, 56.7, 53.5, 53.4, 49.3, 31.6, 31.0, 29.7, 27.7, 23.1, 15.4, 13.9, 7.4; ESI-MS, 846.24 [M+H]⁺.

### Example 14

### Boc-(10)-(7-ethyl-10-hydroxycamptothecin)-(20)-Met-NH₂.HCl (Compound 16)

A solution of Boc-(10)-(7-ethyl-10-hydroxycamptothecin)-(20)-Met-Bsmoc (compound 15, 4.1 g, 4.85 mmol) and 4-piperidinopiperidine (1.06 g, 6.31 mmol) in anhydrous CH₂Cl₂ (200 mL) was stirred at room temperature for 5 hours. This mixture was then washed with 0.1 N HCl (2×40 ml), followed by drying the organic layer over anhydrous MgSO₄. This solution was filtered, and the solvent was removed by vacuum distillation to yield 2.8 g of product with purity of about 97%, determined by HPLC. This product was further purified by trituration with ether (3×20 ml), and then trituration with ethyl acetate (4×20 ml) to yield 1.54 g with purity of 97%. ¹³C NMR (75.4 MHz, CDCl₃) δ: 167.2, 166.5, 156.9, 151.12, 150.9, 149.8, 146.3, 145.9, 145.8, 144.9, 131.3, 127.2, 127.0, 125.1, 119.6, 113.8, 96.7, 84.3, 78.2, 67.0, 60.4, 52.2, 49.4, 31.4, 29.6, 29.1, 27.7, 23.2, 15.1, 13.9, 7.7.

### Example 15

### ^{40k}4arm-PEG-Met-(20)-(7-ethyl-10-hydroxycamptothecin)-(10)-Boc (Compound 17)

To an anhydrous CH₂Cl₂ (80 mL) solution, Boc-(10)-(7-ethyl-10-hydroxycamptothecin)-(20)-Met (compound 16, 1.48 g, 2.25 mmol) and 4arm-PEG-COOH (compound 3, 9.0 g, 0.9 mmol) were added at room temperature. The solution was cooled to 0° C., followed by addition of EDC (0.26 g, 1.35 mmol) and DMAP (0.27 g, 2.25 mmol). The mixture was stirred at 0° C. for 1 hour under N₂. Then it was kept at room temperature overnight. The reaction mixture was diluted with 70 ml of CH₂Cl₂, extracted with 30 ml of 0.1 N HCl/1M NaCl aqueous solution. After the organic layer was dried with MgSO₄, the solvent was evaporated under reduced pressure. The residue was dissolved in 40 mL of CH₂Cl₂, and the crude product was precipitated with ether (300 mL). The wet solid resulting from filtration was dissolved in 270 mL of DMF/IPA at 65° C. The solution was allowed to cool down to room temperature within 2˜3 hours, and the product was precipitated. Then the solid was filtered and washed with ether (2×400 mL). The above crystallization procedure in DMF/IPA was repeated. The wet cake was dried under vacuum below 40° C. overnight to give product of 7.0 g. ¹³C NMR (75.4 MHz, CDCl₃) δ: 169.8, 169.6, 166.5, 156.9, 151.2, 151.1, 149.9, 147.0, 146.6, 145.0, 131.7, 127.1, 126.8, 124.9, 119.7, 113.8, 95.5, 84.1, 70.1, 69.9, 66.9, 50.7, 49.2, 31.5, 31.2, 29.6, 27.6, 23.1, 15.3, 13.9, 7.5.

### Example 16

### ^{40k}4arm-PEG-Met-(20)-(7-ethyl-10-hydroxycamptothecin) (Compound 18)

To a solution of 30% TFA in anhydrous CH₂Cl₂ (100 mL), dimethyl sulfide (2.5 mL) and 4arm-PEG-Met-(20)-(7-ethyl-10-hydroxycamptothecin)-(10)-Boc (compound 17, 6.0 g) were added at room temperature. The mixture was stirred for 3 hours, or until disappearance of starting material was confirmed by HPLC. Solvents were removed as much as possible under vacuum at 35° C. The residues were dissolved in 50 mL of CH₂Cl₂, and the crude product was precipitated with ether (350 ml), and filtered. The wet solid was dissolved in a mixture of DMF/IPA (60/300 mL) at 65° C. The solution was allowed to cool down to room temperature within 2˜3 hours, and the product was precipitated. Then the solid was filtered and washed with ether (2×200 mL). The wet cake was dried under vacuum below 40° C. overnight to give product of 5.1 g. ¹³C NMR (75.4 MHz, CDCl₃) δ :169.7, 166.6, 157.0, 156.3, 148.4, 147.3, 145.0, 144.4, 142.9, 131.5, 128.3, 126.4, 122.5, 118.7, 105.2, 94.7, 78.1, 67.0, 50.7, 49.2, 31.6, 31.3, 29.7, 23.0, 15.3, 13.5, 7.5; Ratio of 7-ethyl-10-hydroxycamptothecin to PEG:2.1% (wt).

### Example 17

### Boc-(10)-(7-ethyl-10-hydroxycamptothecin)-(20)-Sar-Boc (Compound 19)

Boc-Sar-OH (432 mg, 2.287 mmol) was added to a solution of Boc-(10)-(7-ethyl-10-hydroxycamptothecin) (compound 10, 750 mg, 1.52 mmol) in 75 mL of DCM and cooled to 0° C. DMAP (432 mg, 2.287 mmol) and EDC (837 mg, 0.686 mmol) were added and the reaction mixture was stirred from 0° C.-room temperature for 1.5 hours. Reaction mixture was then washed with 0.5% NaHCO₃ (75 mL×2), with water (75 ml×2) and finally washed with 0.1 N HCl (75 mL×1). The methylene chloride layer was dried over MgSO₄ and the solvent was evaporated under vacuum and dried. Yield=0.900 mg. (89%). The structure was confirmed by NMR.

### Example 18

### 7-ethyl-10-hydroxycamptothecin-(20)-Sar.TFA (Compound 20)

Boc-(10)-(7-ethyl-10-hydroxycamptothecin)-(20)-Sar-Boc (compound 19, 900 mg, 1.357 mmol) was added to a solution of 4 mL TFA and 16 mL DCM, and stirred at room temperature for 1 hour. The reaction mixture was evaporated with toluene at 30° C. The residue was dissolved in 10 mL CHCl₃ and precipitated with ethyl ether. The product was filtered and dried. Yield 700 mg (1.055 mmol, 78%). ¹³C NMR (67.8 MHz, CDCl₃) δ 168.26, 167.07, 158.84, 158.71, 148.82, 147.94, 147.22, 146.34, 144.04, 131.18, 130.08, 128.97, 124.46, 119.78, 106.02, 97.23, 79.84, 79.34, 66.87, 50.84, 49.86, 31.81, 23.94, 15.47, 13.84, 8.08.

### Example 19

### TBDMS-(10)-(7-ethyl-10-hydroxycamptothecin)-(20)-Sar.HCl (Compound 21)

A solution of the 7-ethyl-10-hydroxycamptothecin-(20)-Sar.TFA (compound 20, 2.17 g, 3.75 mmol, 1 eq.) in anhydrous DMF (30 mL) was diluted with 200 mL of anhydrous DCM. Et₃N (2.4 mL, 17.40 mmol, 4.5 eq.) was added followed by TBDMSCl (2.04 g, 13.53 mmol, 3.5 eq.). The reaction mixture was stirred at room temperature until HPLC showed disappearance of the starting material (approximately 1 hour). The organic layer was washed with 0.5% NaHCO₃ twice, water once, and a 0.1 N HCl solution saturated with brine twice; and then dried over MgSO₄. After filtration and evaporation of the solvent under vacuum, the resulting oil was dissolved in DCM. Addition of ether gave a solid that was filtered using a fine or medium buchner funnel (2.00 g, 87% yield). HPLC of the solid showed 96% purity. ¹H NMR and ¹³C NMR confirmed the structure. ¹H NMR (300 MHz, CD₃OD): δ 0.23 (6H, s), 0.96 (9H, s), 0.98 (3H, t, J=7.3 Hz), 1.30 (3H, t, J=7.6 Hz), 2.13-2.18 (2H, m), 2.67 (3H, s), 3.11 (2H, q, J=7.6 Hz), 4.10 (1H, d, J=17.6 Hz), 4.22 (1H, d, J=17.6 Hz), 5.23 (2 H, s), 5.40 (1H, d, J=16.7 Hz), 5.55 (1H, d, J=16.7 Hz), 7.32 (1H, s), 7.38-7.43 (2H, m), 8.00 (1H, d, J=9.1 Hz). ¹³C NMR (75.4 MHz, CD₃OD): δ −4.14, 8.01, 14.10, 19.30, 23.98, 26.16, 31.78, 33.52, 49.46, 50.95, 67.66, 79.80, 97.41, 111.96, 119.99, 127.75, 129.28, 129.67, 131.57, 145.24, 146.86, 147.16, 148.02, 150.34, 156.69, 158.72, 167.02, 168.27.

### Example 20

### ^{40K}4arm-PEG-Sar-(20)-(7-ethyl-10-hydroxycamptothecin)-(10)-TBDMS (Compound 22)

To a solution of ^{40K}4-arm-PEG-COOH (compound 3, 10 g, 0.25 mmol, 1 eq.) in 150 mL of anhydrous DCM was added a solution of TBDMS-(10)-(7-ethyl-10-hydroxycamptothecin)-Sar.HCl (compound 21, 1.53 g, 2.5 mmol, 2.5 eq.) in 20 mL of anhydrous DMF and the mixture was cooled to 0° C. To this solution were added EDC (767 mg, 4 mmol, 4 eq.) and DMAP (367 mg, 3 mmol, 3 eq.) and the reaction mixture was allowed to warm to room temperature slowly and stirred at room temperature overnight. Then, the reaction mixture was evaporated under vacuum and the residue was dissolved in a minimum amount of DCM. After addition of ether, solid was formed and filtered under vacuum. The residue was dissolved in 30 mL of anhydrous CH₃CN and precipitated by addition of 600 mL IPA. The solid was filtered and washed with IPA and ether to give the product (9.5 g). The structure was confirmed by NMR.

### Example 21

### ^{40K}4arm-PEG-Sar-(20)-(7-ethyl-10-hydroxycamptothecin) (Compound 23)

Method A. ^{40K}4-arm-PEG-Sar-(20)-(7-ethyl-10-hydroxycamptothecin)-(10) TBDMS (compound 22) was dissolved in a 50% mixture of TFA in H₂O (200 mL). The reaction mixture was stirred at room temperature for 10 hours and then, diluted with 100 mL of H₂O and extracted with DCM (2×300 mL). The combined organic phases were washed with H₂O (2×100 mL), dried over MgSO₄, filtered and evaporated under vacuum. The residue was dissolved in 100 mL of anhydrous DMF gently heated with a heat gun and precipitated by slow addition of 400 mL DMF. The solid was filtered and washed with 20% DMF in IPA and ether. The solid was dissolved in DCM and precipitated with ether (6.8 g). The structure was conformed by NMR.

Method B. ^{40K}4-arm-PEG-Sar-(20)-(7-ethyl-10-hydroxycamptothecin)-(10)-TBDMS (1 g) was dissolved in 10 mL of a 1N HCl solution. The reaction mixture was stirred at room temperature for 1 hour (checked by HPLC) and then extracted with DCM (2×40 mL). The organic layers were dried over MgSO₄, filtered and evaporated under vacuum. The resulting bright yellow residue was dissolved in 10 mL of DMF (slightly heated with a heat gun) and then 40 mL of IPA were added. The resulting solid was filtered and dried overnight at 40° C. in a vacuum oven. The structure was confirmed by NMR.

### Biological Data

### Example 22

### Toxicity Data

A maximum tolerated dose (“MTD”) of four-arm PEG conjugated 7-ethyl-10-hydroxycamptothecin (compound 9) as prepared by Example 7, supra, was studied using nude mice. Mice were monitored for 14 days for mortality and signs of illness and sacrificed when body weight loss was >20% of the pretreatment body weight.

Table 2, below, shows the maximum tolerated dose of each compound for both single dose and multiple dose administration. Each dose for multiple dose administration was given mice every other day for 10 days and the mice were observed for another 4 days, thus for total 14 days.

TABLE 2
MTD Data in Nude Mice

| | Dose Level | Survival/ | |
|---|---|---|---|
| Compound | (mg/kg) | Total | Comments |
| Compound 9 | 25 | 5/5 | |
| Single dose | 30 | 5/5 | |
| | 35 | 4/5 | Mouse euthanized due |
| | | | to >20% body weight loss |
| Compound 9 | 10 | 5/5 | |
| Multiple dose* | 15 | 3/5 | Mice euthanized due |
| | | | to >20% body weight loss |
| | 20 | 0/5 | Mice euthanized due |
| | | | to >20% body weight loss |

The MTD found for 4arm-PEG-Gly-(7-ethyl-10-hydroxycamptothecin) (compound 9) was 30 mg/kg when given as single dose, and 10 mg/kg when given as multiple dose (q2d×5).

### Example 23

### Properties of PEG Conjugates

Table 3, below, shows solubility of four different PEG-(7-ethyl-10-hydroxycamptothecin) conjugates in aqueous saline solution. All four PEG-(7-ethyl-10-hydroxycamptothecin) conjugates showed good solubility of up to 4 mg/mL equivalent of 7-ethyl-10-hydroxycamptothecin. In human plasma, 7-ethyl-10-hydroxycamptothecin was steadily released from the PEG conjugates with a doubling time of 22 to 52 minutes and the release appeared to be pH and concentration dependent as described in the following EXAMPLE 24.

TABLE 3
Properties of PEG-7-ethyl-10-hydroxycamptothecin Conjugates

| | Solubility | t_{1/2}(min) | Doubling Time in | | |
|---|---|---|---|---|---|
| | in Saline | in Human | Plasma (min)^{c} | | |
| Compound | (mg/mL)^{a} | Plasma^{b} | Human | Mouse | Rat |
| Compound 9 | 180 | 12.3 | 31.4 | 49.5 | 570 |
| (Gly) | | | | | |
| Compound 12 | 121 | 12.5 | 51.9 | 45.8 | 753 |
| (Ala) | | | | | |
| Compound 23 | ND | 19.0 | 28.8 | 43.4 | 481 |
| (Sar) | | | | | |
| Compound 18 | 142 | 26.8 | 22.2 | 41.9 | 1920 |
| (Met) | | | | | |

^{a}7-ethyl-10-hydroxycamptothecin is not soluble in saline.
^{b}PEG conjugate half life.
^{c}7-ethyl-10-hydroxycamptothecin formation rate from conjugates.

PEG-7-ethyl-10-hydroxycamptothecin conjugates show good stability in saline and other aqueous medium for up to 24 hours at room temperature.

### Example 24

### Effects of Concentration and pH on Stability

Acylation at the 20-OH position protects the lactone ring in the active closed form. The aqueous stability and hydrolysis properties in rat and human plasma were monitored using UV based HPLC methods. 4armPEG-Gly-(7-ethyl-10-hydroxycamptothecin) conjugates were incubated with each sample for 5 minutes at room temperature.

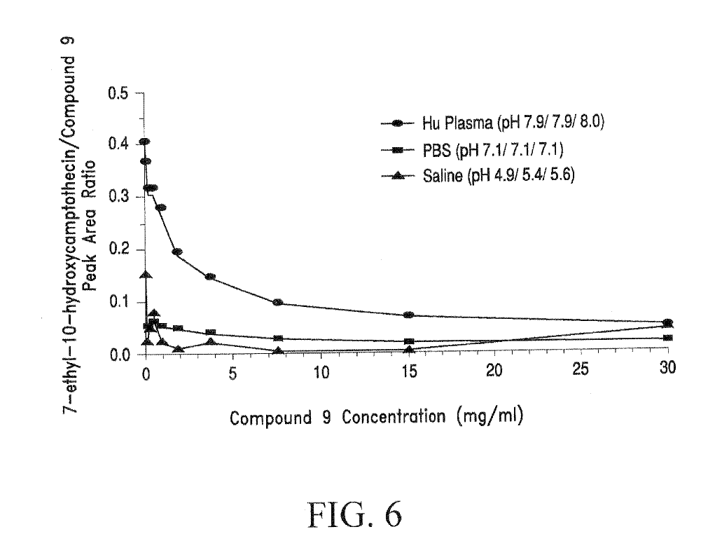

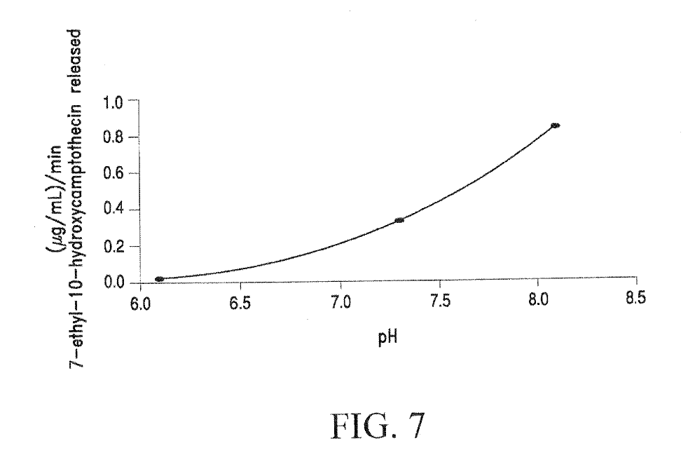

Stability of PEG-7-ethyl-10-hydroxycamptothecin conjugates in buffer was pH dependent. FIG. 6 shows 4armPEG-Gly-(7-ethyl-10-hydroxycamptothecin) stability in various samples. FIG. 7 shows that rate of 7-ethyl-10-hydroxycamptothecin release from PEG-Gly-(7-ethyl-10-hydroxycamptothecin) increases with increased pH.

### Example 25

### Pharmacokinetics

Tumor free Balb/C mice were injected with a single injection of 20 mg/kg 4armPEG-Gly-(7-ethyl-10-hydroxycamptothecin) conjugates. At various time points mice were sacrificed and plasma was analyzed for intact conjugates and released 7-ethyl-10-hydroxycamptothecin by HPLC. Pharmacokinetic analysis was done using non-compartmental analysis (WinNonlin). Details are set forth in Table 4, below.

TABLE 4
Pharmacokinetic Data

| | | 7-ethyl-10-hydroxy- |
|---|---|---|
| | | camptothecin Released |
| Parameter | Compound 9 | from Compound 9 |
| AUC (h*μg/mL) | 124,000 | 98.3 |
| Terminal t_{1/2} (Hr) | 19.3 | 14.2 |
| Cₘₐₓ (μg/mL) | 20,500 | 13.2 |
| CL(mL/hr/kg) | 5.3 | 202 |
| Vss (mL/kg) | 131 | 3094 |

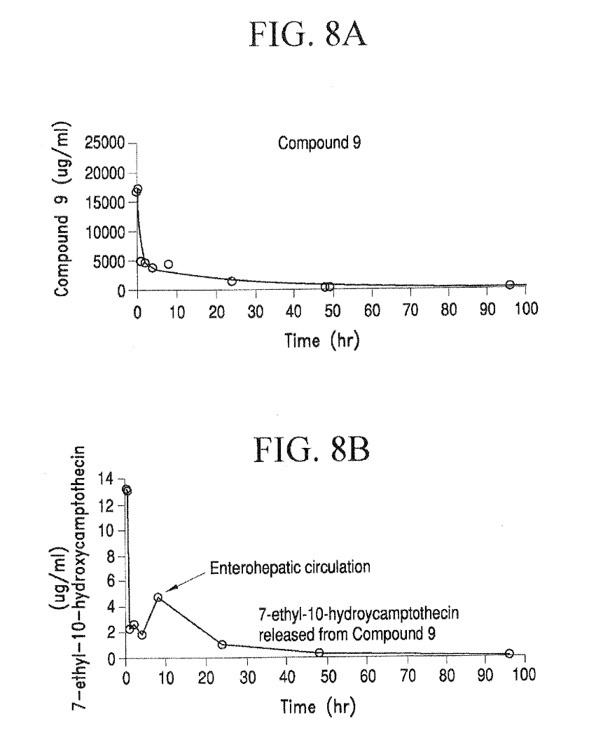

As shown in FIGS. 8A and 8B, PEGylation of 7-ethyl-10-hydroxycamptothecin allows long circulation half life and high exposure to native drug 7-ethyl-10-hydroxycamptothecin. Enterohepatic circulation of 4armPEG-Gly-(7-ethyl-10-hydroxycamptothecin) conjugates was observed. The pharmacokinetic profile of PEG-Gly-(7-ethyl-10-hydroxycamptothecin) in mice was biphasic showing a rapid plasma distribution phase during the initial 2 hours followed by a 18-22 hours terminal elimination half-life for the conjugate and a concomitant 18-26 hours terminal elimination half-life for 7-ethyl-10-hydroxycamptothecin.

Additionally, pharmacokinetic profiles of 4arm PEG-Gly-(7-ethyl-10-hydroxycamptothecin) were investigated in rats. In rats, dose levels of 3, 10 and 30 mg/kg (7-ethyl-10-hydroxycamptothecin equivalent) were used. The pharmacokinetic profiles in rats were consistent with those of mice.

In rats, 4arm PEG-Gly-(7-ethyl-10-hydroxycamptothecin) showed a biphasic clearance from the circulation with an elimination half life of 12-18 hours in rats. 7-ethyl-10-hydroxycamptothecin released from 4armPEG-Gly-7-ethyl-10-hydroxycamptothecin conjugates had an apparent elimination half life of 21-22 hours. The maximum plasma concentration (Cₘₐₓ) and area under the curve (AUC) increased in a dose dependent manner in rats. The apparent half life of released 7-ethyl-10-hydroxycamptothecin from 4armPEG-Gly conjugates in mice or rats is significantly longer than the reported apparent half life of released 7-ethyl-10-hydroxycamptothecin from CPT-11 and the exposure of released 7-ethyl-10-hydroxycamptothecin from 4arm PEG-Gly-(7-ethyl-10-hydroxycamptothecin) is significantly higher than the reported exposure of released 7-ethyl-10-hydroxycamptothecin from CPT-11. The clearance of the parent compound was 0.35 mL/hr/kg in rats. The estimated volume of distribution at steady state (Vss) of the parent compound was 5.49 mL/kg. The clearance of the released 7-ethyl-10-hydroxycamptothecin was 131 mL/hr/kg in rats. The estimated Vss of released 7-ethyl-10-hydroxycamptothecin was 2384 mL/kg in rats. Enterohepatic circulation of released 7-ethyl-10-hydroxycamptothecin was observed both in mice and rats.

### Example 26

### Effects on Angiogenesis—Chorioallantoic Membrane (CAM) Assay

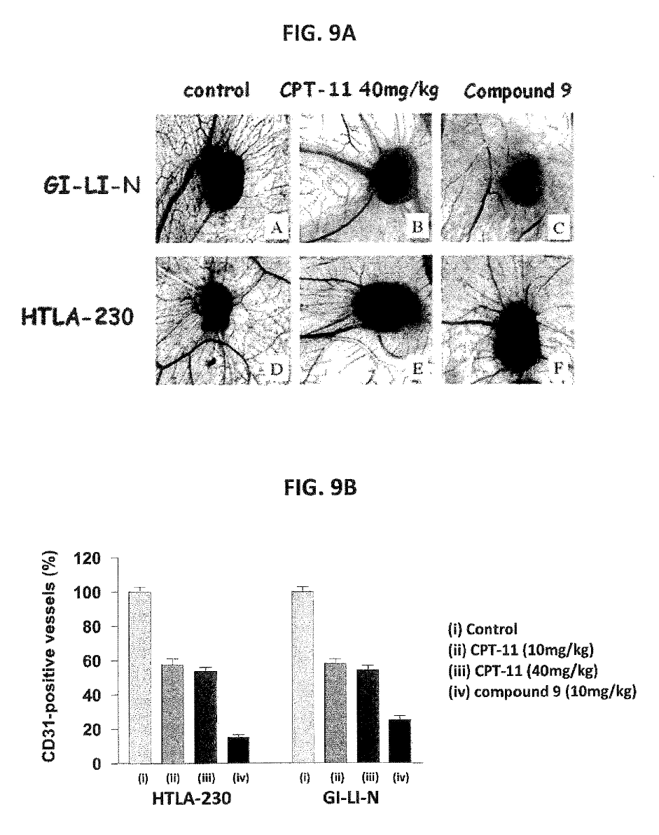

Antiangiogenic activity of compound 9 was evaluated using the CAM assay according to Ribatti D. et al. Nat. Protoc. 2006, 1:85-91. Mice were injected with the human NB cell line, HTLA-230 or GI-LI-N. Tumor biopsy specimens in size of 1-2 mm³ were obtained from the xenografted mice and then grafted onto the CAMs. The CAMs were incubated with CPT-11 at 10 or 40 mg/kg or compound 9 at 10 mg/kg (based on SN38, 7-ethyl-10-hydroxycamptothecin). In the control group, the CAMs were incubated with PBS buffer. In all aspects, the amount of compound 9 is based on the amount of 7-ethyl-10-hydroxycamptothecin, not the amount of polymeric conjugate administered. The CAMs were examined daily for 12 days and photographed in ovo with a stereomicroscope equipped with a camera and image analyzer system (Olympus Italia, Italy). The images are shown in FIG. 9A. CD31-positive microvessels were measured and normalized with that of the control group. Less CD31-positive microvessels mean greater antiangiogenic effects. The results are set forth in FIG. 9B. Microvessel density was represented by the percentage of the total number of intersection points occupied by CD31-positive vessels cut transversely (diameter of 3-10 μm). Mean values±SD were determined for each analysis.

The number of allantoic vessels radiating in a “spoked wheel” pattern towards the tumor specimen was decreased in both CAMs treated with CPT-11 or compound 9, as compared to the control CAMs. The results show that the number of radiating vessels which invade the tumor specimen was much less in the CAMs treated with compound 9 than CPT-11, as shown in (FIGS. 9A and 9B) (P<0.01). The results indicate that compound 9 inhibited angiogenesis significantly as compared to CPT-11.

### Example 27

### Effects on Tumor Cell Angiogenesis and Tumor Invasion in GI-LI-N Xenografted Mice Model

The impacts of compound 9 on angiogenesis and tumor invasion were evaluated in orthotopically implanted human neuroblastoma xenografted mice. Xenograft tumors were established in mice by injecting human neuroblastoma cells (GI-LI-N) in the adrenal gland at day 0 (T₀). Tumors were allowed to grow and reached the average volume of approximately 400 mm³ at day 35 (T₃₅) Then, 10 mg/kg body weight of CAMPTOSAR (CPT-11 in pharmaceutical formulation) or compound 9 (based on SN38) was injected intravenously in the mice at day 35, 37, 39, 41 and 43 (total 5 doses at q2×d). The control group mice received HEPES-buffered saline solution. Histological examination was performed on the tumors removed from the mice at day 44 (T₄₄).

The tissue sections were stained with antibodies against VEGF and CD31 to evaluate inhibition of angiogenesis. The tissue sections were also stained with antibodies against MMP-2 and MMP-9 to detect inhibition of tumor invasion. The antibodies were purchased from the following: anti-VEGF (Thermo Fisher Scientific, Fremont, Calif., USA), and anti-CD31 (clone SC-1506, Santa Cruz Biotechnology, D.B.A Italia S.R.L., Segrate, Milan, Italy), anti-MMP-2 (clone 36006, R & D System, Abingdon, UK) and anti-MMP-9 (clone 443, R & D System). Cell nuclei were stained with DAPI. Morphometric analysis was performed on 9 randomly selected fields every 3 sections, observed at 200× magnification with an Olympus photomicroscope, using Image Analysis software (Olympus Italia). VEGF, CD31, MMP-2, and MMP-9 labelled areas were evaluated. Prior to staining with antibodies against MMP-2 and MMP-9, the paraffin-embedded tissue sections were de-paraffinized by a xylene-ethanol sequence, re-hydrated in a graded ethanol solutions, and TRIS-buffered saline (TBS, pH 7.6), and then processed for antigen retrieval by boiling tissue sections for 10 min in 1 mM EDTA, pH 8.0, in a microwave oven. The sections were then washed twice in PBS and saturated with 2% BSA in PBS. In the morphometric analysis, the mean value in each image from the section, the final mean value for all the images and the SEM were calculated. The statistical significance of the differences between the mean values of the different experimental conditions was determined by Student's t test (GraphPad software). Findings were considered significant at P values <0.05 for all statistical evaluations.

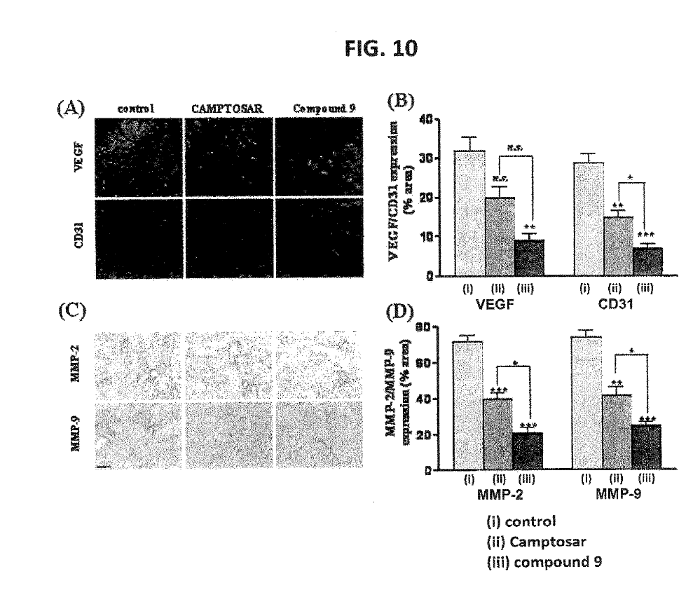

The results are shown in FIGS. 10(A), (B), (C) and (D). The results show that both Camptosar and compound 9 inhibited VEGF and CD31 expression in primary NB tumors. The treatment of mice with compound 9 decreased the number of CD31-positive endothelial cells significantly as compared to the mice treated with CAMPTOSAR. FIG. 10(A). The enhanced inhibition of CD31 expression by the treatment with compound 9 was statistically significant (P<0.05) compared to the treatment with Camptosar. See FIG. 10(B). Compound 9 also inhibited MMP-2 and MMP-9 expression significantly, when compared to Camptosar (P<0.05). FIGS. 10(C) and (D). The errors bars show 95% CI. n.s., not significant; *, p<0.05; **, p<0.01;***, p<0.001.

The results showed that the treatment with compound 9 inhibits tumor angiogenesis and systemic tumor spreading (tumor invasion/metastasis). The results indicate that the treatments described herein have utility in treating patients with a disease associated with angiogenesis such as cancers associated with angiogenesis.

### Example 28

### Effects on Tumor Cell Apoptosis in GI-LI-N Xenografted Mice Model

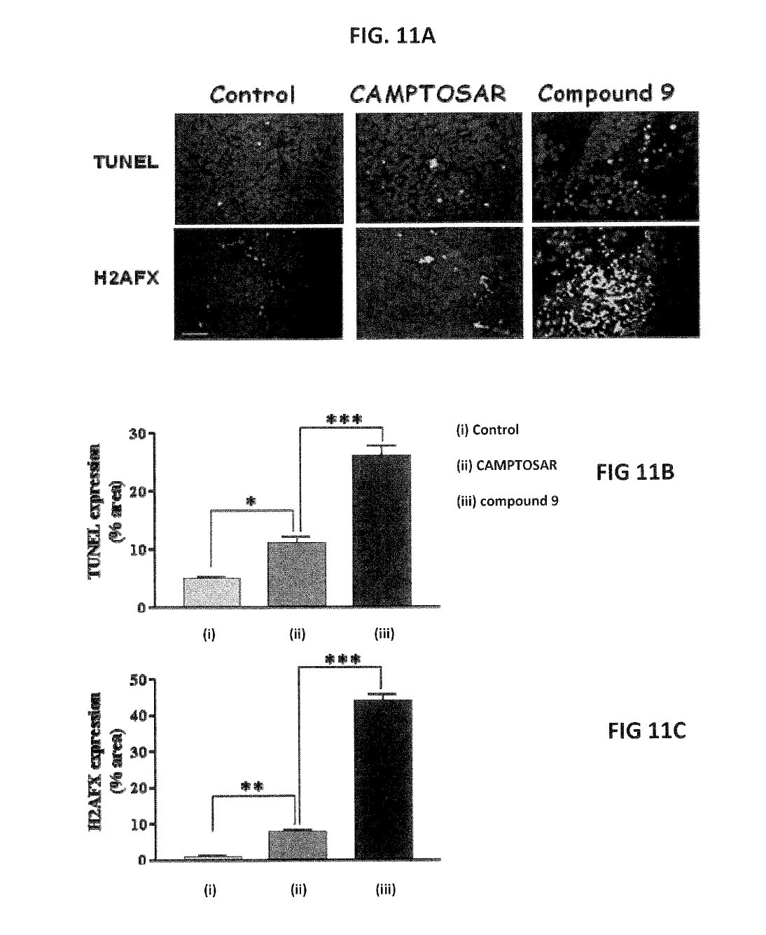

The tissue sections removed from the treated mice in Example 27 were immunostained with TUNEL to evaluate apoptosis, and with primary antibody against histone H2ax (H2AFX) to evaluate DNA-damage dependent histone phosphorylation. The results are shown in FIG. 11 in which scale bars represents 150 μm and error bars show 95% CI. *, p<0.05; **, p<0.01; ***, p<0.001. The results show that enhanced TUNEL and Histone H2ax staining in the tumor tissues removed from the mice treated with compound 9 as compared to the mice treated with CAMPTOSAR. More tumor cells were apoptotic in the mice treated with compound 9 as compared to the mice treated with CPT-11.

### Example 29

### Effects of Compound 9 on HIF-1α Expression in Human Glioma Xenografted Mice Model

The effect of compound 9 on inhibition of HIF-1α expression was evaluated in a human glioma xenograft model. The effect was measured by HIF-1-dependent luciferase expression in the U251-HRE xenografts.

The human glioma cell line, U251-HRE was kindly provided by Dr. Giovanni Melillo at National Cancer Institute (Frederick, Md., United States). The cells were transfected with luciferase reporter plasmids containing three copies of a canonical hypoxia response element (HRE) from the inducible nitric oxide synthase gene (Rapsirada, et al. 2000). U251-HRE tumors were established in the right axillary flank of female Harlan Sprague-Dawley nude mice (Harlan World, Indianapolis, Ind.) by subcutaneous injection of 1×10⁷ U251-HRE cells/mouse. When tumor reached the average volume of 100 mm³, the mice were randomly divided into groups of five mice each and dosed intravenously with saline (qd×10), compound 9 (qd×1 with 30 mg/kg or q2d×3 with 10 mg/kg) or CPT-11 (qd×1 with 80 mg/kg or q2d×3 with 40 mg/kg). At each treatment time point, the tumor volume measurements were recorded and the tumor weights (mg) calculated using mg=[tumor length×(tumor width)]/2. Luciferase expression levels in the U251-HRE induced-tumors were measured using bioluminescence at the 0, 48, and 120 hours following the initiation of drug treatment. To do so, the mice were injected intraperineally with 150 mg/kg of D-luciferin Firefly, potassium salt (Biosynth International, Inc., Itasca, Ill.). After 10 minutes, the mice were anesthetized via isofluorane gas and imaged using the Xenogen IVIS 100 Imaging Station (Xenogen Corp., Alameda, Calif.).

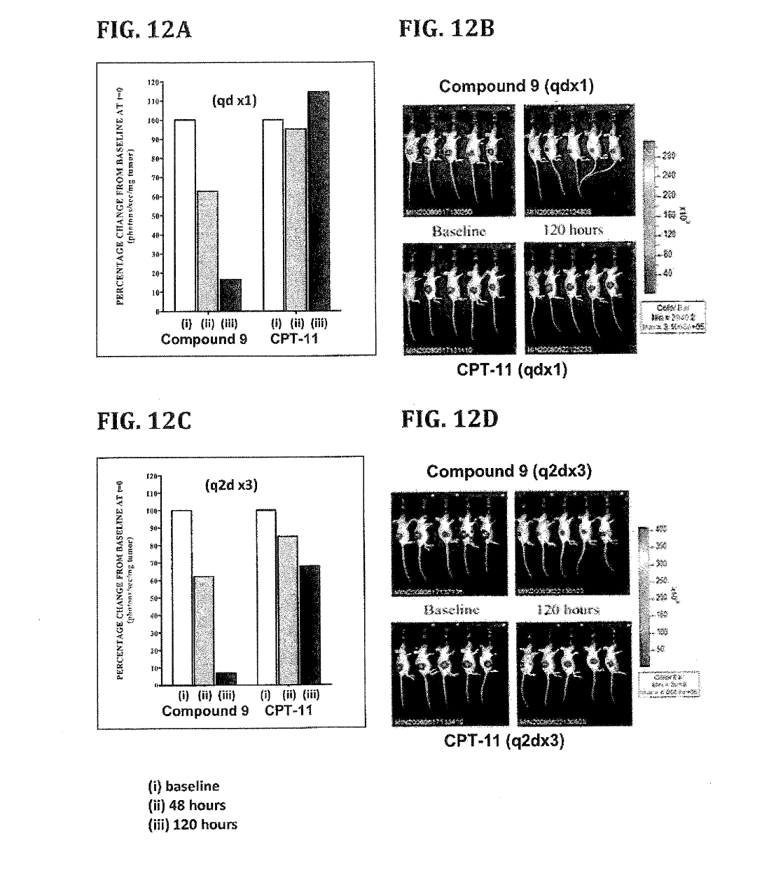

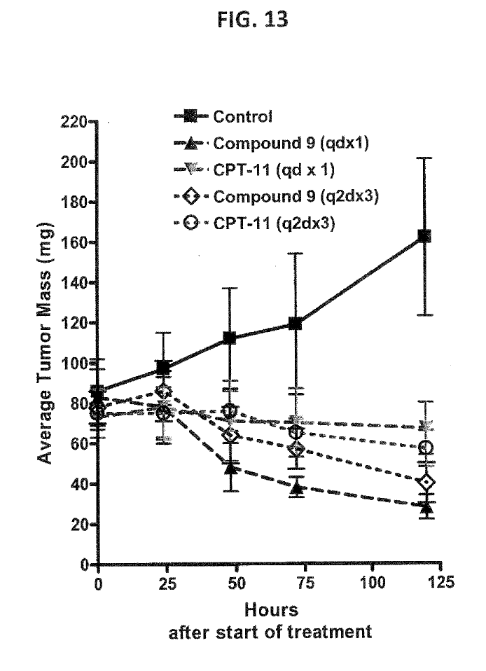

The control mice treated with saline solution had progressive increases in luminescence. The mice treated with the single dose or multiple doses of compound 9 had diminished luminescence at both 48 hour and 120 hour time points (FIGS. 12B and 12D). On the other hand, the CPT-11 treatment had minimal effect on luminescence (FIGS. 12B and 12D). Because the tumor mass was reduced by compound 9 and CPT-11 treatment (FIG. 13), the luminescence values (photons/sec) were normalized for tumor mass and expressed in terms of percent change from baseline (FIGS. 12A and 12C). As seen from FIG. 12A, a single dose of compound 9 induced potent and sustained downregulation of HIF-1α (37% downregulation at 48 hours and 83% downregulation at 120 hours). In contrast, a single injection of CPT-11 caused no downregulation of HIF-1α (FIG. 12A). When given at MTD on multiple days (at day 0, 2, 4), compound 9 induced a very potent downregulation of HIF-1α (93% at 120 hours). In addition, CPT-11 causes a modest 15% and 32% downregulation of HIF-1α at 48 hours and 120 hours, respectively.

The results show that compound 9 downregulated HIF-1α in the human glioma xenograft model and little effect is observed with CPT-11.

### Example 30

### Effects on HIF-1α and HIF-2α Expression

The effects of compound 9 on expression of HIF-1α and HIF-2α in tumor cells were evaluated in vitro using human neuroblastoma cells (GI-LI-N, HTLA-230, and SH-SY5Y).

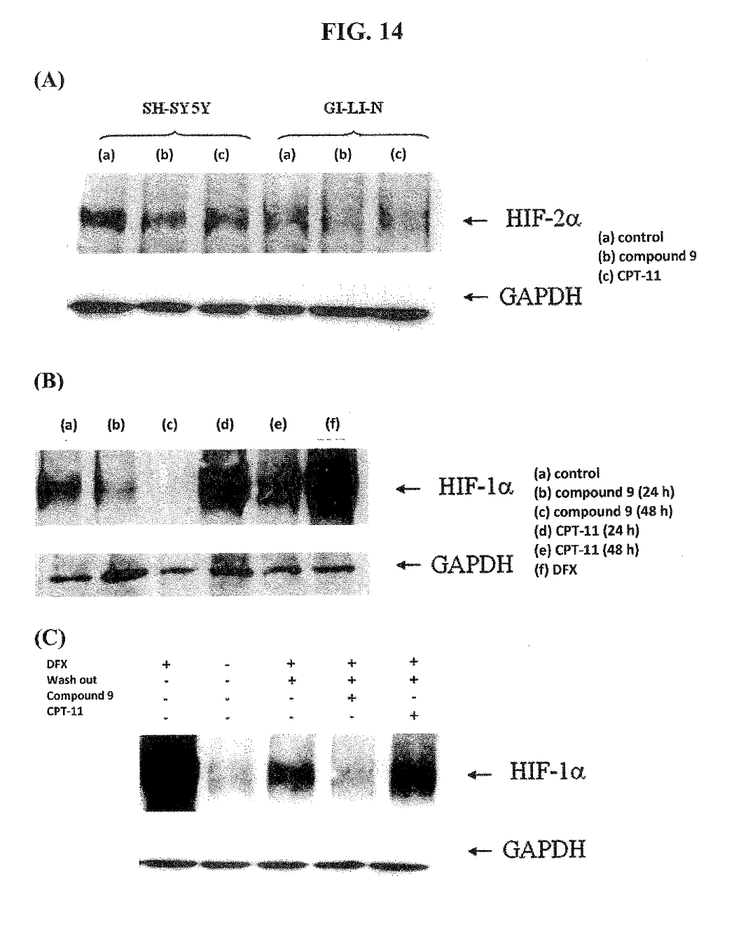

The cancer cells were grown in complete DMEM or RPMI-1640 medium, supplemented with 10% heat-inactivated FCS, as described in Pastorino F. et al., Cancer Res. 2006, 66:10073-82, 2006; Pastorino F. et al., Clin. Cancer Res. 2008, 14:7320-9; and Brignole C, et al., J. Nat'l. Cancer Inst. 2006, 98:1142-57). The cells were treated for 24 and 48 hours with the same concentration of CPT-11 or compound 9 (FIG. 14(A)). In the control, the cells were not treated with CPT-11 and compound 9. In some experiments (FIGS. 14(B) and (C)), the cancer cells were pre-incubated with 0.15 mM of Desferal (DFX or Deferoxamine purchased from Novartis Pharma in Stein, Swizerland) for 6 hours to induce HIF-1α. Thereafter, the cells were washed and treated with CPT-11 and compound 9 for a total of 24 hours. The cells were collected and western blotting analysis was performed using cell lysates as described in Pagnan G. et al., Clin. Cancer Res. 2009, 15:1199-209). Monoclonal anti-p53 (clone PAb 1801) and anti-HIF-1α (clone 54) were purchased from BD Biosciences (Buccinasco, Mich., Italy). Anti-HIF-2α (clone ep190b), and an anti-GAPDH (clone 14c10) antibodies were from Novus Biologicals, Inc (Cambridge, UK) and Cell Signaling Technology (Danvers, Mass., US), respectively.

The results showed that compound 9 inhibited expression of HIF-2α protein. FIG. 14(A). The cell death followed after an rapid and strong induction of p53 (data not shown) and the down regulation of HIF-2α (FIG. 14A). The results also showed that compound 9 decreased both constitutive (FIG. 14B) and DFX-induced (FIG. 14C) HIF-1α protein levels.

The inhibition of HIF-1α expression was significant as compared to CPT-11. The results indicate that compound 9 is potent in inhibiting expression of HIF-1α and HIF-2α.

Sprouting of new blood vessels from preexisting capillaries under the influence of pro-angiogenic growth factor expression, such as VEGF, has been reported. Ribatti D, et al., Eur. J. Cancer, 2002, 38:750-7. HIF-1α mediates angiogenesis by induction of VEGF and plays a role in tumor angiogenesis and invasion. Carmeliet P. et al., Nature, 1998, 394:485-90 and Du R. et al., Cancer Cell 2008, 13:206-20. Without being bound by any theory, the treatment described herein reduces HIF-1α, which leads to an increase of p53 protein, and a statistically significant decrease in factors relevant to angiogenesis and tumor invasion such as CD31, VEGF, MMP-2, and MMP-9. HIF-2α is also strongly correlated with high tumor vascularization. Peng J. et al., Proc. Natl. Acad. Sci. USA, 2000, 97:8386-91. The compounds described herein significantly inhibited HIF-1α, and HIF-2α expression, and the treatment with the compounds described herein provides methods useful for treating a disease associated with angiogenesis.

1. A method of inhibiting angiogenesis or angiogenic activity in a mammal, comprising:
administering an effective amount of a compound of Formula (I): 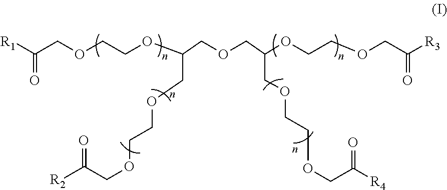
wherein
R₁, R₂, R₃ and R₄ are independently OH or 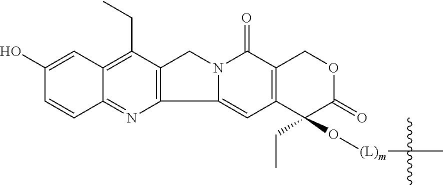
wherein
L is a bifunctional linker;
(m) is 0 or a positive integer, wherein each L is the same or different when (m) is equal to or greater than 2; and
(n) is a positive integer;
provided that R₁, R₂, R₃ and R₄ are not all OH;
or a pharmaceutically acceptable salt thereof to said mammal.

2. The method of claim 1, wherein the angiogenic activity in the mammal is in cells and tissues.

3. The method of claim 1, wherein the angiogenesis is a tumoral angiogenesis or tumor-dependent angiogenesis.

4. The method of claim 1, wherein (n) is from about 28 to about 341 so that the total average molecular weight of the polymeric portion of the compound of Formula (I) ranges from about 5,000 to about 60,000 daltons.

5. The method of claim 4, wherein (n) is from about 114 to about 239 so that the total molecular weight of the polymeric portion of the compound of Formula (I) ranges from about 20,000 to about 42,000 daltons.

6. The method of claim 1, wherein the compound of Formula (I) is selected from the group consisting of

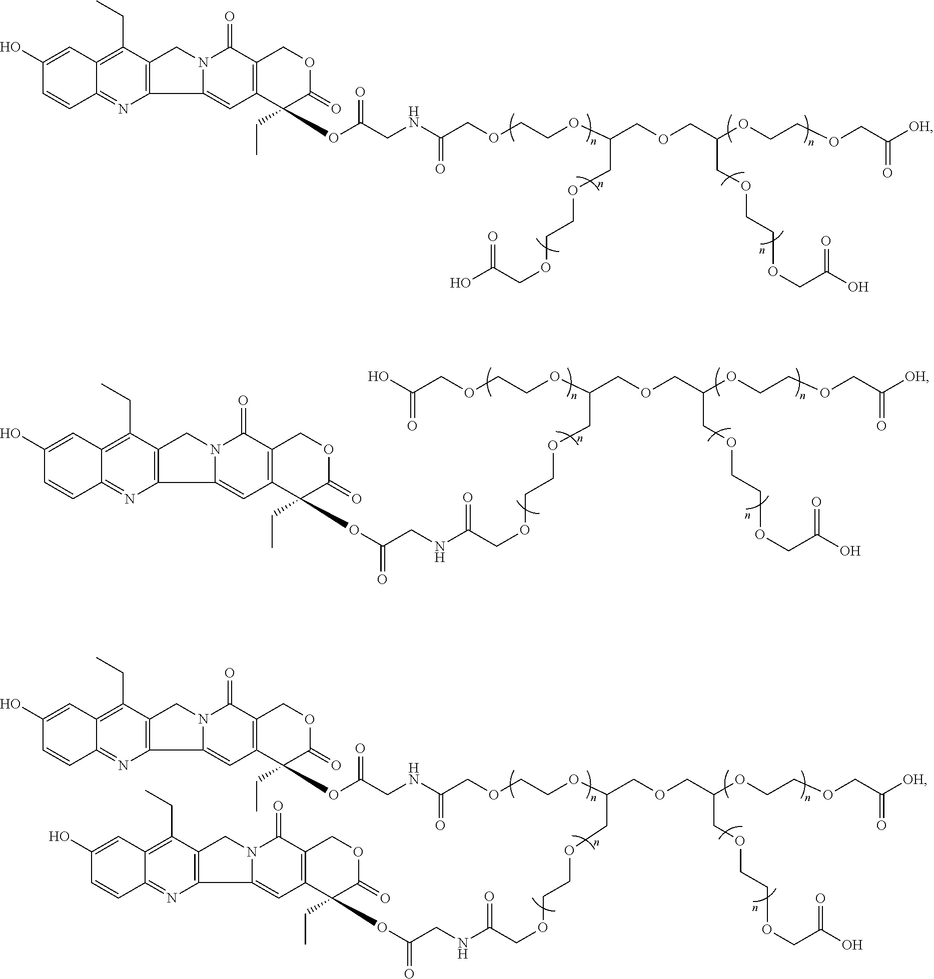

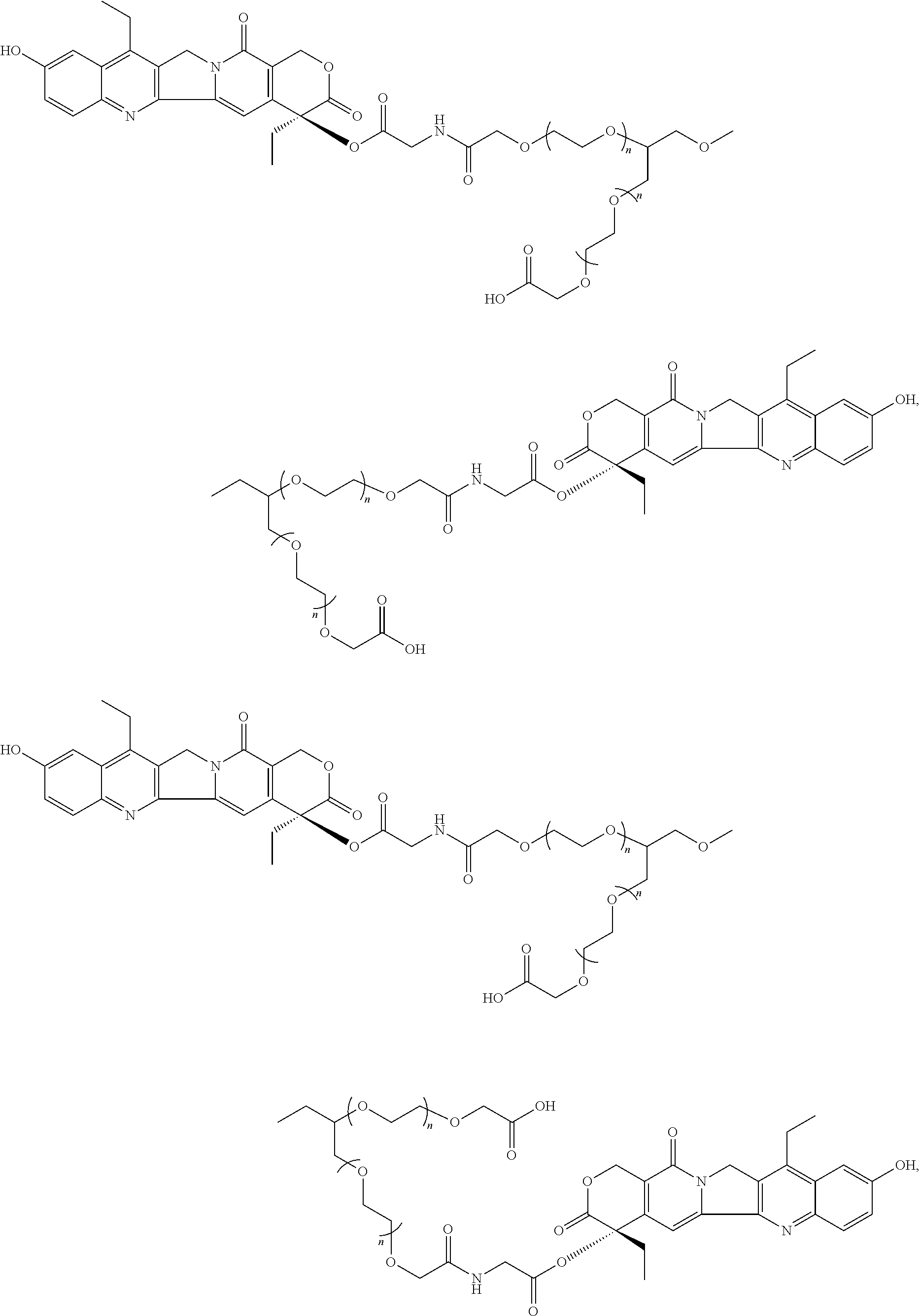

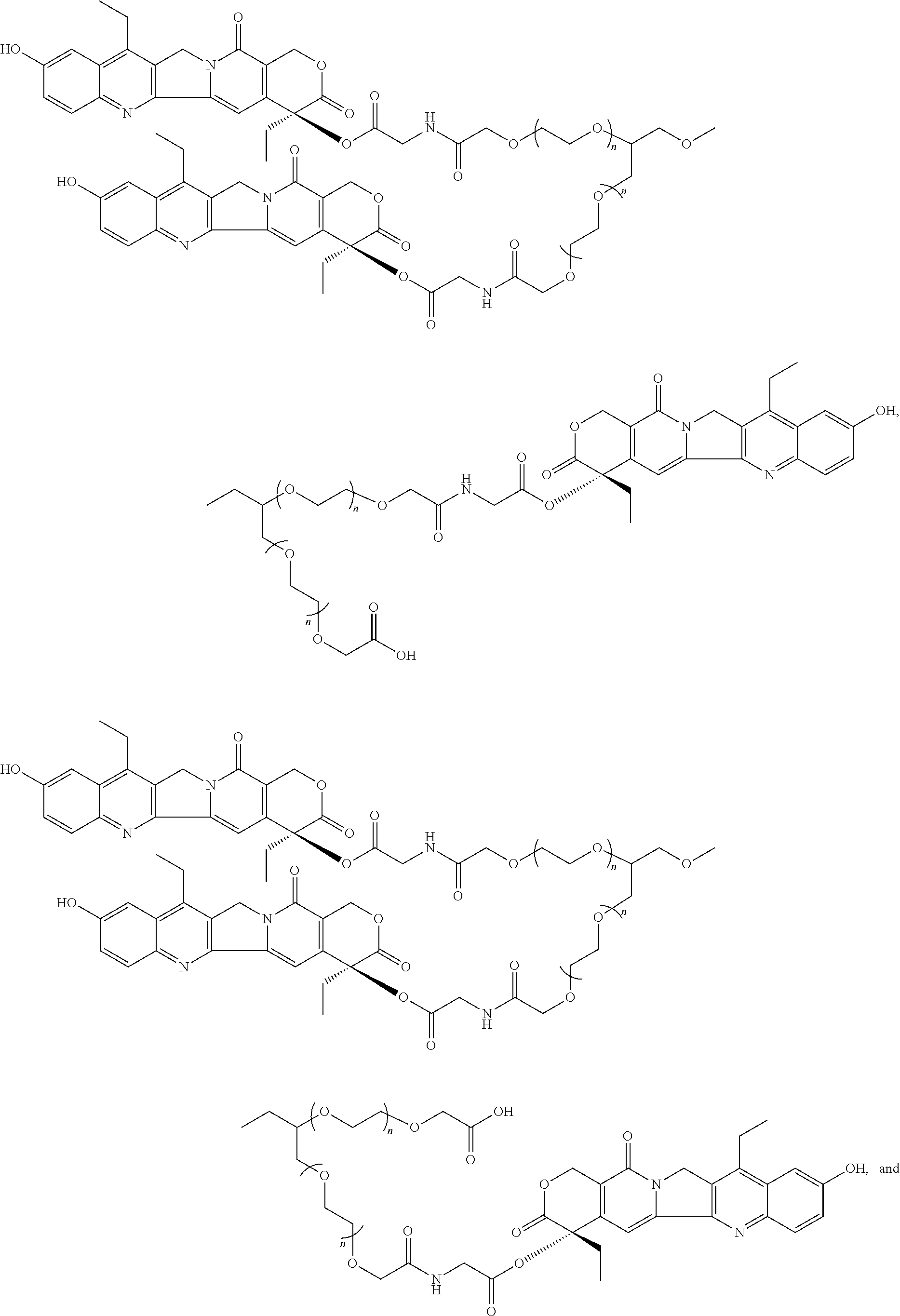

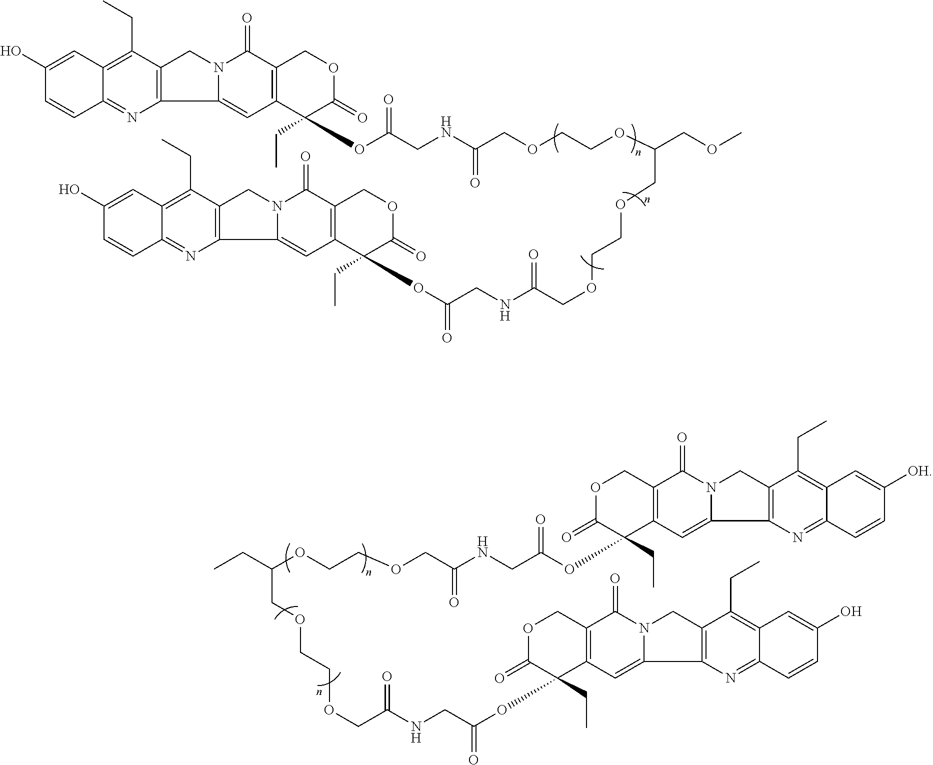

7. The method of claim 1, wherein the compound of Formula (I) is

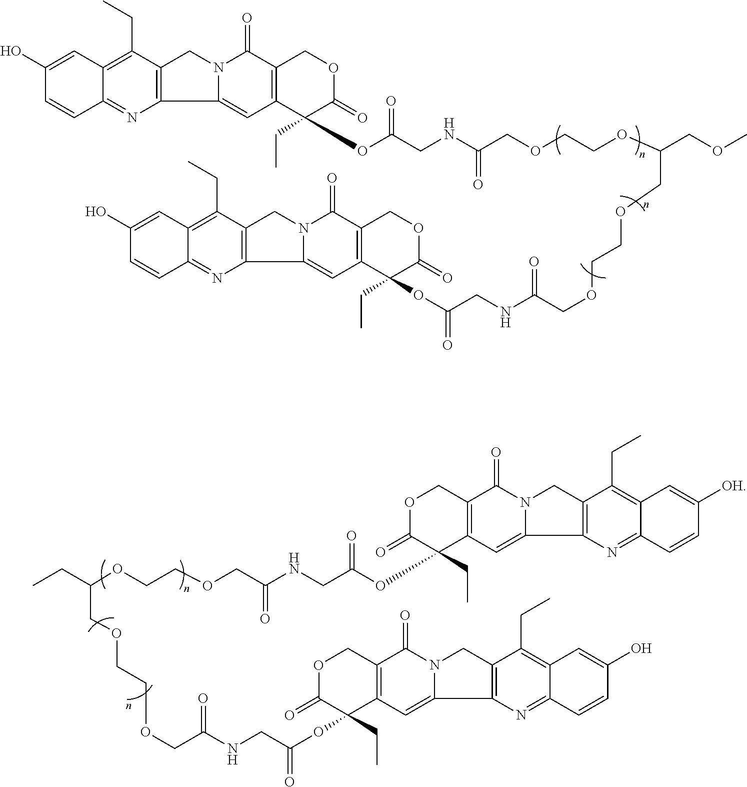

8. The method of claim 1, wherein the compound of Formula (I) is administered in amounts of from about 0.5 mg/m² body surface/dose to about 50 mg/m² body surface/dose, and wherein the amount is the weight of 7-ethyl-10-hydroxycamptothecin included in the compound of Formula (I).

9. The method of claim 1, wherein the compound of Formula (I) or an pharmaceutically acceptable salt thereof is administered in combination with an antisense HIF-1α oligonucleotide or an pharmaceutically acceptable salt thereof concurrently or sequentially.

10. The method of claim 9, wherein the antisense HIF-1α oligonucleotide is complementary to at least 8 consecutive nucleotides of HIF-1α pre-mRNA or mRNA.

11. The method of claim 9, wherein the antisense HIF-1α oligonucleotide comprises from about 8 to 50 nucleotides in length.

12. The method of claim 9, wherein the antisense HIF-1α oligonucleotide comprises nucleotides that are complementary to at least 8 consecutive nucleotides set forth in SEQ ID NO: 1.

13. The method of claim 9, wherein the antisense HIF-1α oligonucleotide comprises one or more phosphorothioate internucleotide linkages.

14. The method of claim 9, wherein the antisense HIF-1α oligonucleotide includes one or more locked nucleic acids (LNA).

15. The method of claim 9, wherein the antisense HIF-1α oligonucleotide is administered in an amount of from about 2 to about 50 mg/kg/dose.

16. A method of inhibiting angiogenesis or angiogenic activity in a mammal, comprising:
administering an effective amount of a compound of 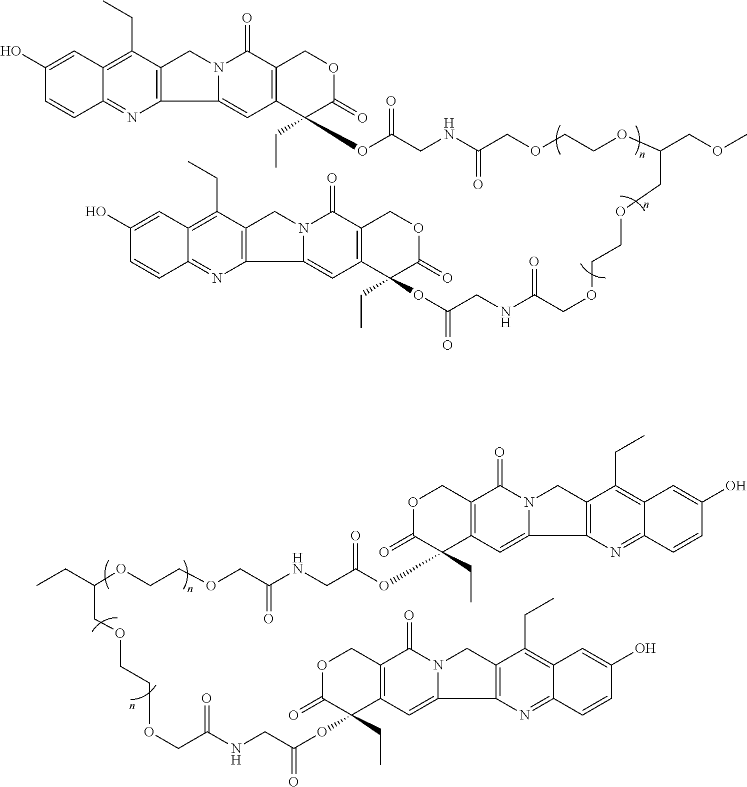
or a pharmaceutically acceptable salt thereof to said mammal
wherein (n) is about 227 so that the total molecular weight of the polymeric portion of the compound of Formula (I) is about 40,000 daltons.

17. (canceled)

18. A method of inhibiting the growth of an angiogenesis-dependent cell, inducing or promoting apoptosis, reducing a vascular network in a mammal having a cancer, or for treating a disease or disorder associated with angiogenesis in a mammal, comprising:
administering an effective amount of a compound of Formula (I): 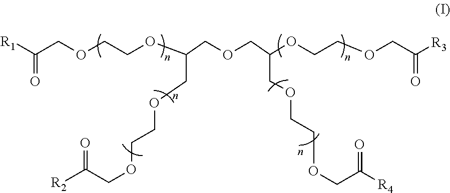
wherein
R₁, R₂, R₃ and R₄ are independently OH or 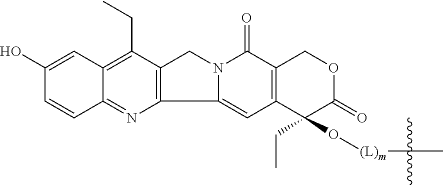
wherein
L is a bifunctional linker;
(m) is 0 or a positive integer, wherein each L is the same or different when (m) is equal to or greater than 2; and
(n) is a positive integer;
provided that R₁, R₂, R₃ and R₄ are not all OH;
or a pharmaceutically acceptable salt thereof to said mammal.

19. The method of claim 18, wherein an antisense HIF-1α oligonucleotide or a pharmaceutically acceptable salt thereof is administered in combination with the compound of Formula (I) or an pharmaceutically acceptable salt thereof concurrently or sequentially.

20. The method of claim 19, wherein the antisense HIF-1α oligonucleotide comprises nucleotides that are complementary to at least 8 consecutive nucleotides set forth in SEQ ID NO: 1, or one or more phosphorothioate internucleotide linkages, and one or more locked nucleic acids (LNA).

21. (canceled)

22. The method of claim 19, wherein the antisense HIF-1α oligonucleotide is administered in an amount of from about 2 to about 50 mg/kg/dose.

23. The method of claim 18, wherein the cell is cancerous cell.

24. (canceled)

25. The method of claim 18, wherein the apoptosis in the mammal is in tumor cells.

26. A method of treating a cancer in a mammal, comprising administering to said mammal
(i) an effective amount of an antisense HIF-1α oligonucleotide of about 8 to 50 nucleotides in length that is complementary to at least 8 consecutive nucleotides set forth in SEQ ID NO: 1 or a pharmaceutically acceptable thereof, wherein the antisense HIF-1α oligonucleotide comprises one or more phosphorothioate internucleotide linkages, and one or more locked nucleic acids; and
(ii) an effective amount of a compound of Formula (Ia) 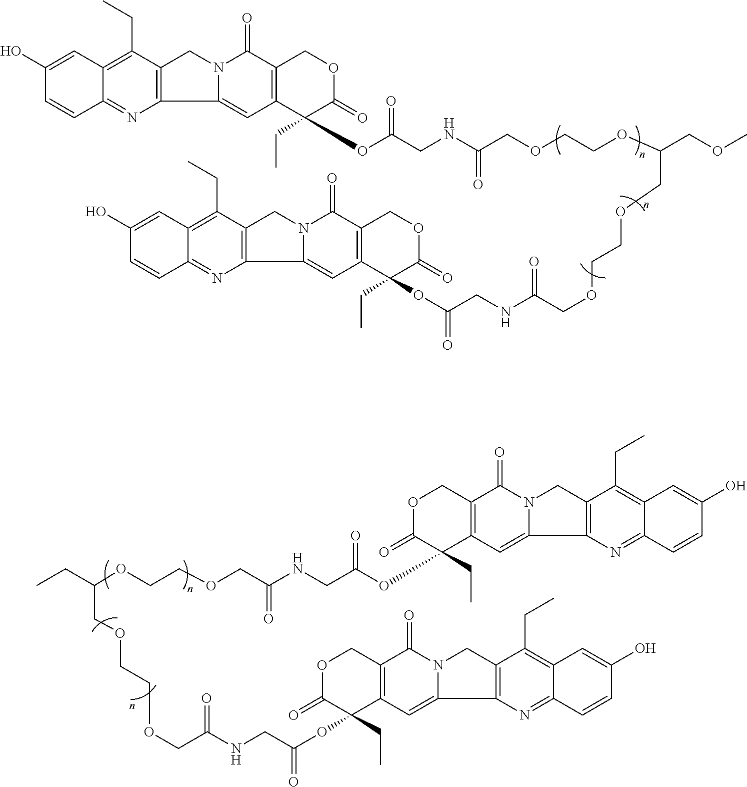
or a pharmaceutically acceptable salt thereof, wherein (n) is about 227 so that the total molecular weight of the polymeric portion of the compound of Formula (Ia) is about 40,000 daltons,
wherein
the antisense HIF-1α oligonucleotide is administered in an amount of from about 4 to about 25 mg/kg/dose, and
the compound of Formula (Ia) is administered in an amount of from about 1 mg/m² body surface/dose to about 18 mg/m² body surface/dose and the amount is the weight of 7-ethyl-10-hydroxycamptothecin included in the compound of Formula (Ia).

27. The method of claim 26, wherein the cancer is an angiogenesis-dependent cancer.

28. (canceled)

29. (canceled)
